(19) United States
(12) Patent Application Publication
(10) Pub. No.: US 2014/0348853 A1
(43) Pub. Date: Nov. 27, 2014

(54) METHODS OF TREATING RICKETTSIA USING EXCHANGE PROTEINS DIRECTLY ACTIVATED BY CAMP (EPACS) INHIBITORS

(71) Applicants: Bin Gong, League City, TX (US); Xiaodong Cheng, League City, TX (US); David Walker, Galveston, TX (US); Fang Mei, League City, TX (US)

(72) Inventors: Bin Gong, League City, TX (US); Xiaodong Cheng, League City, TX (US); David Walker, Galveston, TX (US); Fang Mei, League City, TX (US)

(73) Assignee: The Board of Regents of the University of Texas System, Austin, TX (US)

(21) Appl. No.: 14/455,325

(22) Filed: Aug. 8, 2014

Related U.S. Application Data
(63) Continuation-in-part of application No. 14/377,574.
Application No. 14/377,574 is a continuation-in-part of application No. PCT/US13/25319, filed on Feb. 8, 2013.
(60) Provisional application No. 61/597,369, filed on Feb. 10, 2012.

(51) Int. Cl. A61K 31/42; A61K 45/06

(52) U.S. Cl. CPC A61K 31/42; A61K 45/06
USPC 424/153.1; 514/378; 424/184.1

(57) ABSTRACT

Embodiments of the invention are directed to compounds that inhibit an activity of EPAC proteins and methods of using the same.

### PRIORITY

This application is a continuation in part of and claims priority to U.S. application Ser. No. 14/377,574 filed Aug. 8, 2014, which is a national stage filing of International Application number PCT/US13/25319 filed Feb. 8, 2013, which claims priority to U.S. Provisional Application Ser. No. 61/597,369 filed Feb. 10, 2012. Priority is claimed to the above referenced applications and each application is incorporated herein by reference in its entirety.

### STATEMENT REGARDING FEDERALLY FUNDED RESEARCH

This invention was made with government support under R01GM066170 and R21NS066510 awarded by the National Institute of Health. The United States Government has certain rights in the invention.

### TECHNICAL FIELD

Embodiments of the invention are directed to pharmacology, medicine, and medicinal chemistry. Certain embodiments are directed to methods of treating Rickettsia infection by administering an Exchange Proteins Directly Activated By cAMP (EPAC) inhibitor.

### BACKGROUND

Rickettsioses represent some of the most devastating human infections. Rickettsioses are tick borne diseases, such as typhus fever (Rickettsia prowazekii), caused by obligate intracellular bacteria of the genus Rickettsia, which is a NIAID Category B Priority pathogen. It has been forecast that temperature increases due to global climate change will lead to more widespread incidence of rickettsioses. In addition, a high infectivity and severe illness after inhalation make rickettsiae a potential bioterrorism threat. Although rickettsial infections can be controlled by appropriate broad-spectrum antibiotic therapy if diagnosed early, up to 20% of misdiagnosed or untreated, and 5% of treated Rocky Mountain spotted fever (RMSF) cases result in a fatal outcome. In fact, a fatality rate as high as 32% has been reported in hospitalized patients with Mediterranean spotted fever.

Strains of R. prowazekii resistant to tetracycline and chloramphenicol have been developed in laboratories. Therefore, novel mechanism-based treatments are urgently needed. Cyclic AMP-mediated cell signaling regulates a myriad of important biological processes under both physiological and pathological conditions, and plays crucial roles in the development of many human diseases, including microbial pathogenesis. In eukaryotic cells, the effects of cAMP are transduced by two groups of intracellular cAMP receptors, the classic protein kinase A/cAMP-dependent protein kinase (PKA/cAPK) and a new family of more recently discovered exchange proteins directly activated by cAMP (EPAC) contain an evolutionally conserved cAMP-binding domain, a structural motif that acts as a molecular switch for sensing intracellular second messenger cAMP levels. Depending upon the specific cellular context, EPAC and PKA can act antagonistically or synergistically in controlling various cellular functions.

There remains a need for additional pharmaceuticals and treatment methods for rickettisosis.

### SUMMARY

Certain embodiments are directed to EPAC specific inhibitors as therapeutics targeting EPAC in diseases, such as bacterial infections, where cAMP signaling and EPAC proteins have been implicated.

Certain embodiments are directed to methods for treating Rickettsia infection comprising administering an EPAC inhibitor to a subject having or at risk of having a Rickettsia infection. In certain aspects, the EPAC inhibitor is selected from N-(5-tert-Butyl-isoxazol-3-yl)-2-[(3-chlorophenyl)-hydrazono]-2-cyanoacetamide (HJC0683); 2-[(3-Chlorophenyl)-hydrazono]-2-cyano-N-(5-methyl-isoxazol-3-yl)acetamide (HJC0692); 3-(5-tert-Butyl-isoxazol-3-yl)-2-[(3-chlorophenyl)-hydrazono]-3-oxo-propionitrile (ESI-09); 3-(5-tert-Butyl-isoxazol-3-yl)-2-[(2-chlorophenyl)-hydrazono]-3-oxo-propionitrile (HJC0693); 3-(5-tert-Butyl-isoxazol-3-yl)-2-[(4-chlorophenyl)-hydrazono]-3-oxo-propionitrile (HJC0694); 3-(5-tert-Butyl-isoxazol-3-yl)-3-oxo-2-(phenyl-hydrazono)-propionitrile (HJC0695); 3-(5-tert-Butyl-isoxazol-3-yl)-2-[(2,5-dichlorophenyl)-hydrazono]-3-oxo-propionitrile (HJC0696); 3-(5-tert-Butyl-isoxazol-3-yl)-3-oxo-2-(m-tolyl-hydrazono)propionitrile (HJC0712); 3-(5-tert-Butyl-isoxazol-3-yl)-3-oxo-2-[(3-trifluoromethyl-phenyl)-hydrazono]propionitrile (HJC0720); 3-(5-tert-Butyl-isoxazol-3-yl)-2-[(3-nitrophenyl)-hydrazono]-3-oxo-propionitrile (HJC0721); 3-(5-tert-Butyl-isoxazol-3-yl)-3-oxo-2-(p-tolyl-hydrazono)propionitrile (HJC0724); 3-(5-tert-Butyl-isoxazol-3-yl)-2-[(3,5-dichlorophenyl)-hydrazono]-3-oxo-propionitrile (HJC0726); 2-[(4-Bromophenyl)-hydrazono]-3-(5-tert-butyl-isoxazol-3-yl)-3-oxo-propionitrile (HJC0742); 2-[(3-Bromophenyl)-hydrazono]-3-(5-tert-butyl-isoxazol-3-yl)-3-oxo-propionitrile (HJC0743); 3-(5-tert-Butyl-isoxazol-3-yl)-2-[(2,5-dimethylphenyl)-hydrazono]-3-oxo-propionitrile (HJC0744); 3-(5-tert-Butyl-isoxazol-3-yl)-3-oxo-2-(quinolin-6-yl-hydrazono)propionitrile (HJC0745); 3-(5-tert-Butyl-isoxazol-3-yl)-2-[(2,3-dichlorophenyl)-hydrazono]-3-oxo-propionitrile (HJC0750); 3-(5-tert-Butyl-isoxazol-3-yl)-2-[(3-ethynyl-phenyl)-hydrazono]-3-oxo-propionitrile (HJC0751); 3-{N′-[2-(5-tert-Butyl-isoxazol-3-yl)-1-cyano-2-oxo-ethylidene]-hydrazino}benzoic acid ethyl ester (HJC0752); 3-{N′-[2-(5-tert-Butyl-isoxazol-3-yl)-1-cyano-2-oxo-ethylidene]-hydrazino}benzonitrile (HJC0753); 2-[(3-Acetyl-phenyl)-hydrazono]-3-(5-tert-butyl-isoxazol-3-yl)-3-oxo-propionitrile (HJC0754); 3-(5-tert-Butyl-isoxazol-3-yl)-2-[(2,3-dimethylphenyl)-hydrazono]-3-oxo-propionitrile (HJC0755); 3-(5-tert-Butyl-isoxazol-3-yl)-2-[(3-hydroxymethylphenyl)-hydrazono]-3-oxo-propionitrile (HJC0756); 3-(5-tert-Butyl-isoxazol-3-yl)-2-(indan-5-yl-hydrazono)-3-oxo-propionitrile (HJC0757); 2-[(3,5-Bis-trifluoromethyl-phenyl)-hydrazono]-3-(5-tert-butyl-isoxazol-3-yl)-3-oxo-propionitrile (HJC0758); 2-{N′-[2-(5-tert-Butyl-isoxazol-3-yl)-1-cyano-2-oxo-ethylidene]-hydrazino}-6-chloro-benzoic acid (HJC0759); 3-(5-tert-Butyl-isoxazol-3-yl)-2-[(3-chloro-4-hydroxy-phenyl)-hydrazono]-3-oxo-propionitrile (HJC0760); 2-[(3-Chloro-phenyl)-hydrazono]-3-(5-methyl-isoxazol-3-yl)-3-oxo-propionitrile (HJC0768); or 2-[(3,5-Dichlorophenyl)-hydrazono]-3-(5-methyl-isoxazol-3-yl)-3-oxo-propionitrile (HJC0770). In a further aspect the EPAC inhibitor is 3-(5-tert-Butyl-isoxazol-3-yl)-2-[(3-chlorophenyl)-hydrazono]-3-oxo-propionitrile (ESI-09) or 3-(5-tert-Butyl-isoxazol-3-yl)-2-[(3,5-dichlorophenyl)-hydrazono]-3-oxo-propionitrile (HJC0726). The rickettsia infection can be a Rickettsia prowazekii, Rickettsia typhi, or Rickettsia rickettsii infection. In certain aspects the methods can further comprise administering a second therapeutic agent. The second therapeutic agent can be an antibiotic, an antigen, or a therapeutic antibody.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention.

As used herein, the term “IC₅₀” refers to an inhibitory dose that results in 50% of the maximum response obtained.

The term half maximal effective concentration (EC₅₀) refers to the concentration of a drug that presents a response halfway between the baseline and maximum after some specified exposure time.

The terms “inhibiting,” “reducing,” or “prevention,” or any variation of these terms, when used in the claims and/or the specification includes any measurable decrease or complete inhibition to achieve a desired result.

As used herein, an “inhibitor” as described herein, for example, can inhibit directly or indirectly the activity of a protein. The term “EPAC inhibitor” refers to a compound that decreases the activity of EPAC in a cell. In certain aspects an EPAC inhibitor decreases cancer cell or carcinoma migration by any measurable amount, as compared to such a cell in the absence of such an inhibitor. EPAC inhibitors include EPAC1 inhibitors and/or EPAC2 inhibitors.

As used herein, an “activator” as described herein, for example, can increase the activity of a protein. The term “EPAC activator” refers to a compound that increases the activity of EPAC in a cell. EPAC activators include EPAC1 activators and/or EPAC2 activators.

An “effective amount” of an agent in reference to treating a disease or condition means an amount capable of decreasing, to some extent, a pathological condition or symptom resulting from a pathological condition. The term includes an amount capable of invoking a growth inhibitory, cytostatic and/or cytotoxic effect and/or apoptosis of the cancer or tumor cells.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, etc.).

As used herein, the term “patient” or “subject” refers to a living mammalian organism, such as a human, monkey, cow, sheep, goat, dogs, cat, mouse, rat, guinea pig, or species thereof. In certain embodiments, the patient or subject is a primate. Non-limiting examples of human subjects are adults, juveniles, infants and fetuses.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

The use of the term “or” in the claims is used to mean “and/or” unless explicitly indicated to refer to alternatives only or the alternatives are mutually exclusive, although the disclosure supports a definition that refers to only alternatives and “and/or.”

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Mice lacking Epac1 are protected from rickettsial infection. (A and B) Disease progression (A) and survival (B) of Epac1+/+(WT) (n=12) and Epac1−/−(KO) (n=17) mice were monitored daily for 8 d postinfection following infection with R. australis (R. a) or mock infection. (C) Representative IHC staining of SFG rickettsiae (red) in foci of necrotic infected liver, lung, and testis from WT C57BL/6 (Epac1+/+) and Epac1−/− mice. Rickettsiae (red) were stained using alkaline phosphatase-fast red, whereas nuclei of mouse cells were counterstained with hematoxylin (blue). The light pink color in the periportal hepatocytes and connective tissue of the testis interstitium is nonspecific staining by the alkaline phosphatase-fast red. (Scale bars: 20 μm.) (D) Invasion of endothelium by rickettsiae in multiple organs postinfection. Representative dual-target immunofluorescent (IF) staining of rickettsiae (green) and von Willebrand factor (red) in brain, liver, lung, and testis from exchange protein directly activated by cAMP 1 (Epac1)+/+ mice (n=12). Cell nuclei are counterstained with DAPI (blue). (Scale bars: 10 μm.)

FIG. 2. Rickettsial infection induces increased expression of Epac1 in rickettsial lesions. (A) Representative dual-target IHC staining of rickettsiae (red) and Epac1 (brown) in lung and testis from Epac1+/+ mice (n=12). Rickettsiae and Epac1 were stained using alkaline phosphatase-fast red and peroxidase-DAB, respectively. Nuclei of mouse cells were counterstained with hematoxylin (blue). (B) Representative dual-target IF staining of rickettsiae (green) and Epac1 (red) in brain from an archived pediatric case of fatal RMSF. Nuclei of human cells are counterstained with DAPI (blue). (Scale bars: 20 μm.)

FIG. 3. Epac1 plays critical role in rickettsial attachment and invasion into nonphagocytic host endothelial cells. (A) Representative dual-target IF staining of rickettsiae (red) and Epac1 (green) in frozen section of ex vivo aortic ring prepared from Epac1+/+ and Epac1−/− mice 30 min postinfection with R. australis. Nuclei of mouse cells were counterstained with DAPI (blue). (B) Total bacteria in EIS-09- and vehicle-exposed HUVECs 30 min postinfection with R. australis were enumerated by IF microscopy. The data presented are representative of three independent experiments. The error bar is SD. *P<0.01. (C) Representative dual-target IF staining of rickettsiae (red) and Epac1 (green) in ESI-09- and vehicle-exposed HUVECs 30 min postinfection with R. australis. Nuclei of HUVECs were counterstained with DAPI (blue). (Scale bars: A, 50 μm; C, 10 μm.)

FIG. 4. Inhibition of Epac1 blocks rickettsial attachment and invasion into human umbilical vein endothelial cells (HUVECs). Extracellular (A) and intracellular (B) bacteria in EIS-09- and vehicle-exposed HUVECs 30 min postinfection with Rickettsia australis were enumerated by IF microscopy. The data presented are representative of three independent experiments. The error bar is SD. *P<0.01.

FIG. 5. Epac1 inhibition blocks rickettsial invasion by impeding the expression of Ku70 outside of the nucleus in HUVECs. Representative dual-target IF staining of rickettsiae (red) and Ku70 (green) in ESI-09- and vehicle-exposed HUVECs 30 min postinfection with R. australis. Nuclei of HUVECs were counterstained with DAPI (blue). (Scale bars: 10 μm.)

FIG. 6. Pharmacological inhibition of Epac1 protects WT mice against rickettsial infection. Disease progression (A) and survival (B) of ESI-09-treated (n=11) and vehicle-treated (n=10) Epac1+/+ (WT) mice were monitored daily for 8 d postinfection with R. australis or mock infection. (C) Representative IHC staining of SFG rickettsiae (red) in liver, lung, and testis from ESI-09- and vehicle-treated Epac1+/+ (WT) mice. Rickettsiae were stained using alkaline phosphatase-fast red, whereas nuclei of mouse cells were counterstained with hematoxylin (blue). (Scale bars: 20 μm.)

### DESCRIPTION

cAMP-mediated signaling regulates a myriad of important biological processes under both physiological and pathological conditions. In multi-cellular eukaryotic organisms, the effects of cAMP are transduced by the protein kinase A/cAMP-dependent protein kinase (PKA/cAPK) and the exchange protein directly activated by cAMP/cAMP-regulated guanine nucleotide exchange factor (EPAC/cAMP-GEF) (de Rooij et al. (1998) Nature 396: 474-477; Kawasaki et al. (1998) Science 282: 2275-2279). Since both PKA and EPAC are ubiquitously expressed in all tissues, an increase in intracellular cAMP levels will lead to the activation of both PKA and EPAC. Net physiological effects of cAMP entail the integration of EPAC- and PKA-dependent pathways in a spatial and temporal manner. Depending upon their relative abundance, distribution and localization, as well as the precise cellular environment, the two intracellular cAMP receptors may act independently, converge synergistically, or oppose each other in regulating a specific cellular function (Cheng et al. (2008) Acta Biochim Biophys Sin (Shanghai) 40: 651-662). Therefore, careful dissections of the individual role and relative contribution of EPAC and PKA within the overall cAMP signaling in various model systems are critical for further elucidating the mechanism of cAMP signaling, as well as essential for developing novel mechanism-based therapeutic strategies targeting specific cAMP-signaling components.

Cyclic AMP is a second messenger that induces physiological responses ranging from growth and differentiation to hormonal, neuronal, and immunological regulation (Tasken and Aandahl (2004) Physiol Rev 84:137-167; Holz. (2004) Diabetes 53:5-13). In the brain, it is involved in memory (Huang et al. (1995) Cell 83:1211-1222) and cognitive functions (Sur and Rubenstein (2005) Science 310:805-810). There are two forms of EPAC, EPAC1 and EPAC2, which are encoded by separate genes, EPAC1 and EPAC2, respectively. EPAC1 is expressed ubiquitously with predominant expression in the thyroid, kidney, ovary, skeletal muscle, and specific brain regions. EPAC2 is predominantly expressed in the brain and adrenal gland (de Rooij et al. (1998) Nature 396:474-477 Kawasaki et al. (1998) Science 282:2275-2279).

### I. RICKETTSIA/RICKETTSIOSIS

Rickettsia is a genus of non-motile, Gram-negative, non-spore forming, highly pleomorphic bacteria that can present as cocci (0.1 μm in diameter), rods (1-4 μm long) or thread-like (10 μm long). Being obligate intracellular parasites, the Rickettsia survival depends on entry, growth, and replication within the cytoplasm of eukaryotic host cells (typically endothelial cells). Rickettsia species are carried by chiggers, ticks, fleas, and lice. Rickettsia is classified as three groups (spotted fever, typhus and scrub typhus) based on serology. This grouping has since been confirmed by DNA sequencing. All three of these contain human pathogens. The scrub typhus group has been reclassified as a new genus—Orientia—but many medical textbooks still list this group under the rickettsial diseases.

Species of Rickettsia include, but are not limited to Rickettsia aeschlimannii, Rickettsia africae, Rickettsia akari, Rickettsia asiatica, Rickettsia australis, Rickettsia Canadensis, Rickettsia conorii, Rickettsia cooleyi, Rickettsia felis, Rickettsia heilongjiangensis, Rickettsia Helvetica, Rickettsia honei, Rickettsia hulinii, Rickettsia japonica, Rickettsia massiliae, Rickettsia montanensis, Rickettsia parkeri, Rickettsia peacockii, Rickettsia prowazekii, Rickettsia rhipicephali, Rickettsia rickettsii, Rickettsia sibirica, Rickettsia slovaca, Rickettsia tamurae, and Rickettsia typhi.

Rickettsioses are among the oldest known anthropod-borne diseases. Tick-borne rickettsioses are known in the Americas, Europe, Asia, and Africa. One prominent form of rickettsiosis in the United States is Rocky Mountain spotted fever, caused by infection with Rickettsia rickettsii, which is carried by two or more tick species of the genus Dermacentor. Typical clinical manifestations of rickettsiosis include fever, headache, muscle pain, rash, local lymphadenopathy and other symptoms. Other types of rickettsiosis include, but are not limited to, epidemic typhus, endemic typhus, urban typhus, scrub typhus, recrudescent typhus, Oriental spotted fever, Mexican typhus, Australian tick typhus, Stuttgart disease, European typhus, exanthematous typhus, boutonneuse fever, Manchurian typhus, Mexican typhus, tsutsugamushi disease, rickettsialpox, typhus mitior, North Queensland typhus, Queensland tick typhus, Brill-Zinsser disease, shop typhus and Siberian tick typhus. (Parola and Raoult, Clin. Infect. Dis., 32:897-928 (2001) and Stedman's Medical Dictionary, 26th ed., Williams & Wilkins, Baltimore (1995)). Another embodiment of the invention encompasses the treatment, prevention and/or management of symptoms associated with Rickettsioses.

A. Methods of Treatment Using EPAC Inhibitors

Cyclic adenosine monophosphate (cAMP) is an important component of cell-signaling networks that control numerous biological processes. More than a decade of extensive studies have now firmly established that many cAMP-related cellular processes, previously thought to be controlled by PKA alone, are also mediated by EPAC (Gloerich and Bos, (2010) Annu Rev Pharmacol Toxicol 50:355-375). For example, EPAC proteins have been implicated in regulating exocytosis and secretion (Ozaki et al. (2000) Nat Cell Biol 2:805-811; Seino and Shibasaki (2005) Physiol Rev 85:1303-1342; Maillet et al. (2003) Nat Cell Biol 5:633-639; Li et al. (2007) Mol Endocrinol 21:159-171), cell adhesion (Enserink et al. (2004) J Biol Chem 279:44889-44896; Rangarajan et al. (2003) J Cell Biol 160:487-493), endothelial barrier junctions (Cullere et al. (2005) Blood 105:1950-1955; Kooistra et al. (2005) FEBS Lett 579:4966-4972), leptin signaling, and cardiac functions (Metrich et al. (2010) Pflugers Arch 459:535-546). In addition to its regulatory functions under physiological conditions, cAMP has been implicated in playing a major role in multiple human diseases, including cancer, diabetes, heart failure, and neurological disorders, such as Alzheimer's disease (AD). The EPAC1 and/or EPAC2 modulating compounds described herein can be used to provide treatment for a variety of diseases or conditions associated with EPAC activation or inhibition.

In certain aspects EPAC specific inhibitors can be used for attenuating or preventing uptake of a microbe by a vascular endothelial cell. Endothelial and epithelial cell-cell junctions and barriers play a critical role in the dissemination of microbe infection. EPAC and its down-stream effector Rap1 have been shown to play an important role in cellular functions related to endothelial cell junctions and barrier (Kooistra et al. (2005) FEBS Lett 579:4966-4972; Baumer et al. (2009) J Cell Physiol. 220:716-726; Noda et al. (2010) Mol Biol Cell 21:584-596; Rampersad et al. J. Biol Chem. 285:33614-33622; Spindler et al (2011) Am J Pathol 178:2424-2436). In addition, EPAC is known to be involved in phagocytosis (Yeager et al (2009) Infect Immun 77:2530-2543; Shirshev (2011) Biochemistry (Mosc) 76:981-998).

Cyclic AMP is a universal second messenger that is evolutionally conserved in diverse form of lives, including human and pathogens such as bacterial, fungi and protozoa. It has been well recognized that cAMP play major roles in microbial virulence, ranging from a potent toxin to a master regulator of virulence gene expression. (MaDonough & Rodriguez (2012) Nature Rev Microbiol 10:27-38). As a major intracellular cAMP receptor, it is likely that EPAC proteins are important cellular targets for microbe infection.

To determine if EPAC1 plays a role in rickettsia infection, WT and EPAC1^{-/-} C57BL/6 mice were challenged with sublethal dose of R. australia. All WT mice became severely ill 5 days post infection and a few WT mice died. On the other hand, none of the EPAC1^{-/-} mice became severely sick. These results suggest that deletion of EPAC 1 protects mice from R. australia infection.

To test if EPAC inhibitors are capable of protecting mice from lethal-dose infection of R. australia. WT C57BL/6 mice were treated with vehicle or ESI-09 (10 mg/kg, IP) daily. Five days after the treatment, mice were challenged with lethal dose of R. australia and continued ESI-09 daily treatment. Similar to EPAC1 genetic deletion, pharmacological inhibition of EPAC1 also led to a striking protection of R. Australia infection. 100% control group became severely sick while only 10% of the treatment group showed sign of sickness.

To investigate the mechanism of EPAC1 inhibition-mediated protection of R. australia infection, HUVEC cells treated with vehicle or ESI-09 were infected with R. australia. The number of intracellular R. australia was dramatically reduced in ESI-09 treated HUVEC cells. These data demonstrate that inhibition of EPAC by ESI-09 treatment suppresses cellular entry of R. australia.

Certain embodiments are directed to methods of suppressing microbe infection comprising administering an EPAC specific inhibitor to a subject having or under the risk of microbe infection. In certain aspects the microbe is a bacteria, virus, or fungi. In other aspects the EPAC specific inhibitor is selected from the EPAC inhibitors described herein.

B. EPAC Inhibitors

Certain embodiments are directed to an isolated Exchange Protein Activated by cAMP (EPAC) modulating compound having a general formula of Formula I:

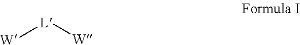

where L′ is —SO₂—, —NH—, or —C(O)—C(CN)═N—NH—; and W′ and W″ are independently substituted or unsubstituted aryl, or substituted or unsubstituted heteroaryl.

Further embodiments are directed to an isolated Exchange Protein Activated by cAMP (EPAC) modulating compound having a general formula of Formula II:

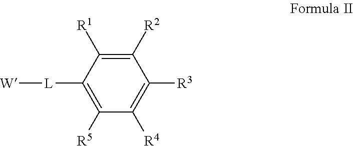

where R¹, R², R³, R⁴, and R⁵ are independently hydrogen, hydroxyl, halogen, C₁-C₄ alkoxy; substituted or unsubstituted C₁-C₁₀ alkyl, substituted or unsubstituted C₁-C₁₀ heteroalkyl, substituted or unsubstituted C₅-C₇ cycloakyl, substituted or unsubstituted C₅-C₇ heterocyclyl, substituted or unsubstituted aryl, substituted or unsubstituted heteroaryl, or C₁-C₅, alkylamine; L is —SO₂— or —NH—; and W′ is as described above for Formula I. In a further aspect, L is —SO₂—. In certain aspects W′ is substituted phenyl or N-containing heteroaryl. In yet another aspect, a nitrogen in the N-containing heteroaryl is attached to L.

An isolated Exchange Protein Activated by cAMP (EPAC) modulating compound having a general formula of Formula III:

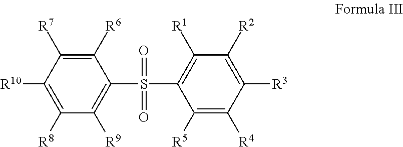

where R¹, R², R³, R⁴, R⁵, R⁶, R⁷, R⁸, R⁹, and R¹⁰ are independently hydrogen, hydroxyl, halogen, C₁-C₄ alkoxy, substituted or unsubstituted C₁-C₁₀ alkyl, substituted or unsubstituted C₁-C₁₀ heteroalkyl, substituted or unsubstituted C₅-C₇ cycloakyl, substituted or unsubstituted C₅-C₇ heterocyclyl, substituted or unsubstituted aryl, substituted or unsubstituted heteroaryl, or C₁-C₅, alkylamine. In certain aspects R¹, R², R³, R⁴, R⁵, R⁶, R⁷, R⁸, R⁹, and R¹⁰ are independently hydrogen or C₁-C₁₀ alkyl. In a further aspect, R¹, R³, and R⁵ are C₁-C₁₀ alkyl; and R² and R⁴ are hydrogen. In still further aspects, one or more of R⁷, R⁹, and R¹⁰ are C₁-C₁₀ alkyl. In yet further aspects R⁷, R⁹, and R¹⁰ are C₁-C₁₀ alkyl. In certain aspects R¹⁰ is substituted or unsubstituted C₁-C₄ alkyl or C₁-C₄ alkoxy. In yet other aspects, R¹⁰ is halide or halo-substituted heteroaryl.

Certain embodiments are directed to a compound of Formula III where R¹, R³, and R⁵ are methyl; R² and R⁴ are hydrogen; and (a) R⁷, R⁹, and R¹⁰ are C₁-C₁₀ alkyl, and R⁶ and R⁸ are hydrogen; (b) R¹⁰ is C₁-C₁₀ alkyl, and R⁶, R⁷, R⁸, R⁹ are hydrogen; (c) R¹⁰ is C₁-C₄ alkoxy, and R⁶, R⁷, R⁸, R⁹ are hydrogen; (d) R¹⁰ is halogen, and R⁶, R⁷, R⁸, R⁹ are hydrogen; (e) R¹⁰ is hydroxyl, and R⁶, R⁷, R⁸, R⁹ are hydrogen; or (f) R¹⁰ is a halogen or C₁₋₄ alkyl substituted pyridine, or a 2-, 4-, 5-, or 6-halo-pyridine, and R⁶, R⁷, R⁸, R⁹ are hydrogen.

Certain embodiments are directed to a compound of Formula III where R¹, R³, and R⁵ are methyl; R² and R⁴ are hydrogen; and (a) R⁷, R⁹, and R¹⁰ are methyl, and R⁶ and R⁸ are hydrogen; (b) R¹⁰ is methyl, and R⁶, R⁷, R⁸, R⁹ are hydrogen; (c) R¹⁰ is methoxy, and R⁶, R⁷, R⁸, R⁹ are hydrogen; (d) R¹⁰ is iodo, and R⁶, R⁷, R⁸, R⁹ are hydrogen; (e) R¹⁰ is hydroxyl, and R⁶, R⁷, R⁸, R⁹ are hydrogen; or (f) R¹⁰ is 5-fluoro-pyridine and R⁶, R⁷, R⁸, R⁹ are hydrogen.

Certain embodiments are directed to a compound of Formula III where R³ is methyl; R¹, R², R⁴, and R⁵, are hydrogen; and (a) R⁷, R⁹, and R¹⁰ are C₁-C₁₀ alkyl, and R⁶ and R⁸ are hydrogen; (b) R¹⁰ is C₁-C₁₀ alkyl, and R⁶, R⁷, R⁸, R⁹ are hydrogen; (c) R¹⁰ is C₁-C₄ alkoxy, and R⁶, R⁷, R⁸, R⁹ are hydrogen; (d) R¹⁰ is halogen, and R⁶, R⁷, R⁸, R⁹ are hydrogen; (e) R¹⁰ is hydroxyl, and R⁶, R⁷, R⁸, R⁹ are hydrogen; or (f) R¹⁰ is a halogen, C₁₋₄ alkyl substituted pyridine, or a 2-, 4-, 5-, or 6-halo-pyridine, and R⁶, R⁷, R⁸, R⁹ are hydrogen.

Certain embodiments are directed to a compound of Formula III where R³ is methyl; R¹, R², R⁴, and R⁵, are hydrogen; and (a) R⁷, R⁹, and R¹⁰ are methyl, and R⁶ and R⁸ are hydrogen; (b) R¹⁰ is methyl, and R⁶, R⁷, R⁸, R⁹ are hydrogen; (c) R¹⁰ is methoxy, and R⁶, R⁷, R⁸, R⁹ are hydrogen; (d) R¹⁰ is iodo, and R⁶, R⁷, R⁸, R⁹ are hydrogen; (e) R¹⁰ is hydroxyl, and R⁶, R⁷, R⁸, R⁹ are hydrogen; or (f) R¹⁰ is 5-fluoro-pyridine, and R⁶, R⁷, R⁸, R⁹ are hydrogen.

In certain embodiments the compound of formula III is 1,3,5-trimethyl-2-(2,4,5-trimethyl-bensenesulfonyl)-benzene (HJC-2-71); 2-(4-methoxy-benzenesulfonyl)-1,3,5-trimethyl-benzene (HJC-2-82); 1,3,5-Trimethyl-2-(toluene-4-sulfonyl)-benzene (HJC-2-85); 4-(2,4,6-Trimethyl-benzenesulfonyl)-phenol (HJC-2-87); 2-(4-Iodo-benzenesulfonyl)-1,3,5-trimethyl-benzene (HJC-2-93); 2-Fluoro-5-[4-(2,4,6-trimethyl-benzenesulfonyl)-phenyl]-pyridine (HJC-2-97); or 1,2,4-Trimethyl-5-(toluene-4-sulfonyl)-benzene (HJC-2-98).

Still a further embodiment is directed to an isolated Exchange Protein Activated by cAMP (EPAC) modulating compound having a general formula of Formula IV:

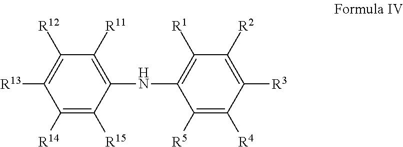

where R¹, R², R³, R⁴, and R⁵ are as described for Formula III above; and R¹¹, R¹², R¹³, R¹⁴, and R¹⁵ are independently hydrogen, halogen, C₁-C₁₀ alkyl, or C₁-C₁₀ heteroalkyl. In certain aspects, R¹, R³, and R⁵ are C₁-C₁₀ alkyl; and R² and R⁴ are hydrogen. In a further aspect, R¹¹, R¹², R¹³, R¹⁴, and R¹⁵ are independently hydrogen, halogen, or C₁-C₁₀ alkyl.

Certain embodiments are directed to compounds of Formula IV where R¹, R³, and R⁵ are C₁-C₁₀ alkyl; R² and R⁴ are hydrogen; and (a) R¹¹ and R¹⁴ are halogen, and R¹², R¹³, and R¹⁵ are hydrogen; (b) R¹² and R¹⁴ are halogen, and R¹¹, R¹³, and R¹⁵ are hydrogen; or (c) R¹³ is C₁-C₁₀ alkyl, and R¹¹, R¹², R¹⁴, and R¹⁵ are hydrogen.

Certain embodiments are directed to compounds of Formula IV where R¹, R³, and R⁵ are methyl; R² and R⁴ are hydrogen; and (a) R¹¹ and R¹⁴ are chloro, and R¹², R¹³, and R¹⁵ are hydrogen; (b) R¹² and R¹⁴ are chloro, and R¹¹, R¹³, and R¹⁵ are hydrogen; or (c) R¹³ is methyl, and R¹¹, R¹², R¹⁴, and R¹⁵ are hydrogen.

In certain aspect the compound of formula IV is (3,5-Dichloro-phenyl)-(2,4,6-trimethyl-phenyl)-amine (HJC-2-83); p-Tolyl-(2,4,6-trimethyl-phenyl)-amine (HJC-2-89); or (2,5-Dichloro-phenyl)-(2,4,6-trimethyl-phenyl)-amine (HJC-3-38).

Certain embodiments are directed to an isolated Exchange Protein Activated by cAMP (EPAC) modulating compound having a general formula of Formula V:

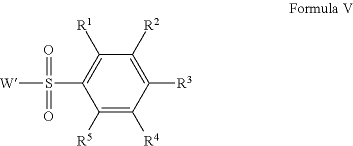

where R¹, R², R³, R⁴, and R⁵ are as described in Formula III above; and W′ is as described in Formula I above. In certain aspects, R¹, R², R³, R⁴, and R⁵ are independently hydrogen, halogen, C₁-C₁₀ alkyl, or C₁-C₁₀ heteroalkyl. In certain aspects, W′ is substituted or unsubstituted indole, substituted or unsubstituted azaindole, or substituted or unsubstituted pyrrole. In certain aspects, W′ is unsubstituted indole or unsubstituted azaindole. In a further aspect, W′ is pyrrole substituted with one or more C₁-C₁₀ alkyl groups. In certain aspects, W′ is 1-ethylpyrrole or 2,4-dimethylpyrrole.

Certain embodiments are directed to compounds of Formula V where R¹, R³, and R⁵ are C₁-C₁₀ alkyl; R² and R⁴ are hydrogen; and W′ is substituted or unsubstituted indole, substituted or unsubstituted azaindole, or substituted or unsubstituted pyrrole. In certain aspects, W′ is unsubstituted indole or unsubstituted azaindole. In a further aspect, W′ is pyrrole substituted with one or more C₁-C₁₀ alkyl groups. In certain aspects, W′ is 1-ethylpyrrole or 2,4-dimethylpyrrole.

Certain embodiments are directed to compounds of Formula V where R¹, R³, and R⁵ are methyl; R² and R⁴ are hydrogen; and W′ is substituted or unsubstituted indole, substituted or unsubstituted azaindole, or substituted or unsubstituted pyrrole. In certain aspects, W′ is unsubstituted indole or unsubstituted 4-, 5-, 6-, or 7-azaindole. In a further aspect, W′ is pyrrole substituted with one or more methyl or ethyl. In certain aspects, W′ is 1-ethylpyrrole or 2,4-dimethylpyrrole.

Certain embodiments are directed to compounds of Formula V where R′ and R³ are C₁-C₁₀ alkyl; R², R⁴, and R⁵ are hydrogen; and W′ is substituted or unsubstituted indole, substituted or unsubstituted azaindole, or substituted or unsubstituted pyrrole. In certain aspects, W′ is unsubstituted indole or unsubstituted azaindole. In a further aspect, W′ is pyrrole substituted with one or more C₁-C₁₀ alkyl groups. In certain aspects, W′ is 1-ethylpyrrole or 2,4-dimethylpyrrole.

Certain embodiments are directed to compounds of Formula V where R¹ and R³ are methyl; R², R⁴, and R⁵ are hydrogen; and W′ is substituted or unsubstituted indole, substituted or unsubstituted azaindole, or substituted or unsubstituted pyrrole. In certain aspects, W′ is unsubstituted indole or unsubstituted 4-, 5-, 6-, or 7-azaindole. In a further aspect, W′ is pyrrole substituted with one or more methyl or ethyl. In certain aspects, W′ is 1-ethylpyrrole or 2,4-dimethylpyrrole.

Certain embodiments are directed to compounds of Formula V where R² and R⁴ are C₁-C₁₀ alkyl; R¹, R³, and R⁵ are hydrogen; and W′ is substituted or unsubstituted indole, substituted or unsubstituted azaindole, or substituted or unsubstituted pyrrole. In certain aspects, W′ is unsubstituted indole or unsubstituted azaindole. In a further aspect, W′ is pyrrole substituted with one or more C₁-C₄ alkyl groups. In certain aspects, W′ is 1-ethylpyrrole or 2,4-dimethylpyrrole.

Certain embodiments are directed to compounds of Formula V where R² and R⁴ are methyl; R¹, R³, and R⁵ are hydrogen; and W′ is substituted or unsubstituted indole, substituted or unsubstituted azaindole, or substituted or unsubstituted pyrrole. In certain aspects, W′ is unsubstituted indole or unsubstituted 4-, 5-, 6-, or 7-azaindole. In a further aspect, W′ is pyrrole substituted with one or more methyl or ethyl. In certain aspects, W′ is 1-ethylpyrrole or 2,4-dimethylpyrrole.

Certain embodiments are directed to compounds of Formula V where R³ is C₁-C₁₀ alkyl; R¹, R², R⁴, and R⁵ are hydrogen; and W′ is substituted or unsubstituted indole, substituted or unsubstituted azaindole, or substituted or unsubstituted pyrrole. In certain aspects, W′ is unsubstituted indole or unsubstituted azaindole. In a further aspect, W′ is pyrrole substituted with one or more C₁-C₁₀ alkyl groups. In certain aspects, W′ is 1-ethylpyrrole or 2,4-dimethylpyrrole.

Certain embodiments are directed to compounds of Formula V where R³ is methyl; R¹, R², R⁴, and R⁵ are hydrogen; and W′ is substituted or unsubstituted indole, substituted or unsubstituted azaindole, or substituted or unsubstituted pyrrole. In certain aspects, W′ is unsubstituted indole or unsubstituted 4-, 5-, 6-, or 7-azaindole. In a further aspect, W′ is pyrrole substituted with one or more methyl or ethyl. In certain aspects, W′ is 1-ethylpyrrole or 2,4-dimethylpyrrole.

Certain embodiments are directed to compounds of Formula V where R¹ is C₁-C₁₀ alkyl; R², R³, R⁴, and R⁵ are hydrogen; and W′ is substituted or unsubstituted indole, substituted or unsubstituted azaindole, or substituted or unsubstituted pyrrole. In certain aspects, W′ is unsubstituted indole or unsubstituted azaindole. In a further aspect, W′ is pyrrole substituted with one or more C₁-C₁₀ alkyl groups. In certain aspects, W′ is 1-ethylpyrrole or 2,4-dimethylpyrrole.

Certain embodiments are directed to compounds of Formula V where R¹ is methyl; R², R³, R⁴, and R⁵ are hydrogen; and W′ is substituted or unsubstituted indole, substituted or unsubstituted azaindole, or substituted or unsubstituted pyrrole. In certain aspects, W′ is unsubstituted indole or unsubstituted 4-, 5-, 6-, or 7-azaindole. In a further aspect, W′ is pyrrole substituted with one or more methyl or ethyl. In certain aspects, W′ is 1-ethylpyrrole or 2,4-dimethylpyrrole.

In certain embodiments the compound of Formula V is 1-(2,4,6-Trimethyl-benzenesulfonyl)-1H-indole (HJC-2-77); 2-Ethyl-1-(2,4,6-trimethyl-benzenesulfonyl)-1H-pyrrole (HJC-2-79); 1-(2,4,6-Trimethyl-benzenesulfonyl)-1H-pyrrolo[2,3-b]pyridine (HJC-2-81); 1-(2,4,6-Trimethyl-benzenesulfonyl)-1H-pyrrolo[2,3-c]pyridine (HJC-3-21); 1-(2,4,6-Trimethyl-benzenesulfonyl)-1H-pyrrolo[3,2-c]pyridine (HJC-3-22); 1-(2,4,6-Trimethyl-benzenesulfonyl)-1H-pyrrolo[3,2-b]pyridine (HJC-3-23); 2-Ethyl-1-(toluene-4-sulfonyl)-1H-pyrrole (HJC-3-26); 2,4-Dimethyl-1-(2,4,6-trimethyl-benzenesulfonyl)-1H-pyrrole (HJC-3-50); 2-Ethyl-1-(toluene-2-sulfonyl)-1H-pyrrole (HJC-3-53); 1-(3,5-Dimethyl-benzenesulfonyl)-2-ethyl-1H-pyrrole (HJC-3-54); 1-(2,4-Dimethyl-benzenesulfonyl)-2-ethyl-1H-pyrrole (HJC-3-55); or 1-(2,4,6-Trimethyl-benzenesulfonyl)-1H-indole-5-carboxylic acid (HJC-3-62).

Certain embodiments are directed to an isolated Exchange Protein Activated by cAMP (EPAC) modulating compound having a formula of:

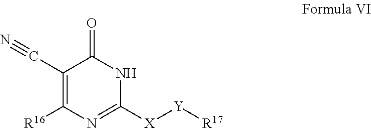

where R¹⁶ is substituted or unsubstituted C₁-C₁₀ alkyl, substituted or unsubstituted C₃-C₆ heteroalkyl, substituted or unsubstituted C₃-C₆ cycloalkyl, substituted or unsubstituted C₃-C₆ heterocyclyl, substituted or unsubstituted aryl, or substituted or unsubstituted heteroaryl; R¹⁷ is hydrogen, substituted or unsubstituted aryl, or substituted or unsubstituted heteroaryl; X is sulfur or nitrogen; and Y is a direct bond, —CH₂—, —CH₂C(O)O—, or —CH₂C(O)N—. Formula VI represents an alternative embodiment of Formula I, where W′ is a substituted pyrimidine, and L is a particular linker designated by —X—Y—.

Certain embodiments are directed to compounds of Formula VI where X is sulfur; Y is —CH₂—; R¹⁶ is as described above for Formula VI; and R¹⁷ is as described above for Formula VI. In certain aspects R¹⁷ is as described above for Formula VI; and R¹⁶ is (a) C₃-C₆ cycloakyl, (b) C₆ cycloakyl, (c) C₅ cycloalkyl, (d) C₄ cycloalkyl, (e) C₃ cycloalkyl, (f) branched or linear C₁-C₁₀ alkyl, or (g) branched C₃ alkyl. In certain aspects, R¹⁷ is substituted phenyl. In certain aspects, R¹⁷ is a C₁-C₁₀ alkyl substituted phenyl. In further aspects, the substituted phenyl has 1, 2, or 3 C₁-C₁₀ alkyl substituents. In certain aspects the C₁-C₁₀ alkyl substituents are at positions 1, 3, and 5; 2 and 5; 2 and 4; 1 and 3; or 3 of the phenyl group. In a further aspect, R¹⁷ is 3,6-dimethylphenyl; 3,5-dimethylphenyl; or 2,4-dimethylphenyl. In yet a further aspect, R¹⁷ is 2,4,6-trimethylphenyl.

Certain embodiments are directed to compounds of Formula VI where X is sulfur; Y is —CH₂C(O)N—; R¹⁶ is as described above for Formula VI; and R¹⁷ is as described above for Formula VI. In certain aspects R¹⁷ is as described above for Formula VI; and R¹⁶ is (a) C₃-C₆ cycloakyl, (b) C₆ cycloakyl, (c) C₅ cycloalkyl, (d) C₄ cycloalkyl, (e) C₃ cycloalkyl, (f) branched or linear C₁-C₁₀ alkyl, or (g) branched C₃ alkyl. In certain aspects, R¹⁷ is substituted phenyl. In certain aspects, R¹⁷ is a C₁-C₁₀ alkyl substituted phenyl. In further aspects, the substituted phenyl has 1, 2, or 3 C₁-C₁₀ alkyl substituents. In certain aspects the C₁-C₁₀ alkyl substituents are at positions 1, 3, and 5; 2 and 5; 2 and 4; 1 and 3; or 3 of the phenyl group. In a further aspect, R¹⁷ is 3,6-dimethylphenyl; 3,5-dimethylphenyl; or 2,4-dimethylphenyl. In yet a further aspect, R¹⁷ is 2,4,6-trimethylphenyl.

Certain embodiments are directed to compounds of Formula VI where X is nitrogen; Y is —CH₂—; R¹⁶ is as described above for Formula VI; and R¹⁷ is as described above for Formula VI. In certain aspects R¹⁷ is as described above for Formula VI; and R¹⁶ is (a) C₃-C₆ cycloakyl, (b) C₆ cycloakyl, (c) C₅ cycloalkyl, (d) C₄ cycloalkyl, (e) C₃ cycloalkyl, (f) branched or linear C₁-C₁₀ alkyl, or (g) branched C₃ alkyl. In certain aspects, R¹⁷ is substituted phenyl. In certain aspects, R¹⁷ is a C₁-C₁₀ alkyl substituted phenyl. In further aspects, the substituted phenyl has 1, 2, or 3 C₁-C₁₀ alkyl substituents. In certain aspects the C₁-C₁₀ alkyl substituents are at positions 1, 3, and 5; 2 and 5; 2 and 4; 1 and 3; or 3 of the phenyl group. In a further aspect, R¹⁷ is 3,6-dimethylphenyl; 3,5-dimethylphenyl; or 2,4-dimethylphenyl. In yet a further aspect, R¹⁷ is 2,4,6-trimethylphenyl.

Certain embodiments are directed to compounds of Formula VI where X is nitrogen; Y is a direct bond; R¹⁶ is as described above for Formula VI; and R¹⁷ is as described above for Formula VI. In certain aspects R¹⁷ is as described above for Formula VI; and R¹⁶ is (a) C₃-C₆ cycloakyl, (b) C₆ cycloakyl, (c) C₅ cycloalkyl, (d) C₄ cycloalkyl, (e) C₃ cycloalkyl, (f) branched or linear C₁-C₁₀ alkyl, or (g) branched C₃ alkyl. In certain aspects, R¹⁷ is substituted phenyl. In certain aspects, R¹⁷ is a C₁-C₁₀ alkyl substituted phenyl. In further aspects, the substituted phenyl has 1, 2, or 3 C₁-C₁₀ alkyl substituents. In certain aspects the C₁-C₁₀ alkyl substituents are at positions 1, 3, and 5; 2 and 5; 2 and 4; 1 and 3; or 3 of the phenyl group. In a further aspect, R¹⁷ is 3,6-dimethylphenyl; 3,5-dimethylphenyl; or 2,4-dimethylphenyl. In yet a further aspect, R¹⁷ is 2,4,6-trimethylphenyl.

In certain embodiments a compound of Formula VI is 4-Cyclohexyl-2-(2,5-dimethyl-benzylsulfanyl)-6-oxo-1,6-dihydro-pyrimidine-5-carbonitrile (HJC-1-65); 4-Cyclohexyl-2-(4-methyl-benzylsulfanyl)-6-oxo-1,6-dihydro-pyrimidine-5-carbonitrile (HJC-1-67); 4-Cyclohexyl-2-(3,5-dimethyl-benzylsulfanyl)-6-oxo-1,6-dihydro-pyrimidine-5-carbonitrile (HJC-1-72); 4-Cyclohexyl-2-(2,4-dimethyl-benzylsulfanyl)-6-oxo-1,6-dihydro-pyrimidine-5-carbonitrile (HJC-1-74); 2-Benzylsulfanyl-4-cyclohexyl-6-oxo-1,6-dihydro-pyrimidine-5-carbonitrile (HJC-1-76); 4-Cyclohexyl-6-oxo-2-(2,4,6-trimethyl-benzylsulfanyl)-1,6-dihydro-pyrimidine-5-carbonitrile (HJC-1-87); 2-(2,5-Dimethyl-benzylsulfanyl)-4-isopropyl-6-oxo-1,6-dihydro-pyrimidine-5-carbonitrile (HJC-1-95); 4-Cyclopentyl-2-(2,5-dimethyl-benzylsulfanyl)-6-oxo-1,6-dihydro-pyrimidine-5-carbonitrile (HJC-1-97); 4-Cyclopropyl-2-(2,5-dimethylbenzylsulfanyl)-6-oxo-1,6-dihydro-pyrimidine-5-carbonitrile (HJC-1-98); 4-Cyclohexyl-6-oxo-2-phenylamino-1,6-dihydro-pyrimidine-5-carbonitrile (HJC-1-99); 4-[5-Cyano-2-(2,5-dimethylbenzylsulfanyl)-6-oxo-1,6-dihydro-pyrimidin-4-yl]-piperidine-1-carboxylic acid tert-butyl ester (HJC-1-93); (5-Cyano-4-cyclohexyl-6-oxo-1,6-dihydro-pyrimidin-2-ylsulfanyl)-acetic acid (HJC-2-4); 2-(5-Cyano-4-cyclohexyl-6-oxo-1,6-dihydro-pyrimidin-2-ylsulfanyl)-N-(2,4,6-trimethyl-phenyl)-acetamide (HJC-3-33); or 2-(5-Cyano-4-cyclohexyl-6-oxo-1,6-dihydro-pyrimidin-2-ylsulfanyl)-N-p-tolyl-acetamide (HJC-3-35).

Certain embodiments are directed to an isolated Exchange Protein Activated by cAMP (EPAC) modulating compound having a formula of:

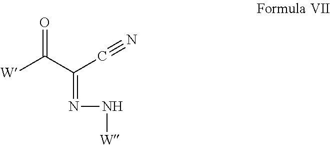

in certain aspects W′ and W″ are as described for Formula I above.

In certain embodiments W′ is an unsubstituted or substituted isoxazole. In certain aspects the isoxazole is attached via the 3 position. In certain aspects the substituted isoxazole is a 4-substituted isoxazole, a 5-substituted isoxazole, or a 4,5-substituted isoxazole. In a particular aspect the substituted isoxazole is a 5-substituted isoxazole. In certain aspects the substituent is independently a branched or unbranched C₁ to C₁₀ alkyl. In certain aspect the alkyl is a methyl, ethyl, n-propyl, iso-propyl, n-butyl, sec-butyl, iso-butyl, tert-butyl, neo-pentyl, n-pentyl, or isopenyl. In certain embodiments the isoxazole is a 5-methyl or 5 tert-butyl isoxazole. In a further aspect W′ can be a substituted to unsubstituted phenyl.

In certain embodiments W″ is a monocyclic or polycyclic, substituted or unsubstituted aryl or heteroaryl. In certain aspects W″ is a substituted phenyl or N-containing heteroaryl. In a further aspect the substituted phenyl is a 2; 3; 4; 5; 6; 2,3; 2,4; 2,5; 2,6; 3,4; 3,5; 3,6; 4,5; 4,6; or 5,6 substituted phenyl. In still further aspects the phenyl comprises one or more substituent selected from bromo, fluoro, chloro, iodo, C₁-C₄ alkyl, hydroxy, nitro, fluoromethyl, difluoromethyl, trifluoromethyl, nitrile, C₁-C₄ alkynyl, acetyl, C₁-C₄ hydroxyalkyl, C₁-C₄ alkoxy, or carboxyl group. In certain aspects W″ is a substituted or unsubstituted benzopyridine or a substituted or unsubstituted indane. In certain aspects W″ is a 3-chlorophenyl; 2-chlorophenyl; 4-chlorophenyl; phenyl; 3,6-dichlorophenyl; 3-methylphenyl, 3-trifluoromethylphenyl; 3-nitrophenyl; 4-methylphenyl, 3,5-dichlorophenyl; 4-bromophenyl; 3-bromophenyl; 3,6-dimethylphenyl; benzopyridine; 2,3-dichlorophenyl; 3-ethynyl; benzoic acid ethyl ester; 3-benzonitrile; 3-acetylphenyl; 2,3-methylphenyl; 3-ethoxyphenyl; indane; 3,5-di-trifluoromethylphenyl; 6-chloro-benzoic acid; or 3-chloro, 4-hydroxyphenyl.

In certain aspects a compound of Formula VII is selected from N-(5-tert-Butyl-isoxazol-3-yl)-2-[(3-chlorophenyl)-hydrazono]-2-cyanoacetamide (HJC0683); 2-[(3-Chlorophenyl)-hydrazono]-2-cyano-N-(5-methyl-isoxazol-3-yl)acetamide (HJC0692); 3-(5-tert-Butyl-isoxazol-3-yl)-2-[(3-chlorophenyl)-hydrazono]-3-oxo-propionitrile (HJC0680, ESI-09); 3-(5-tert-Butyl-isoxazol-3-yl)-2-[(2-chlorophenyl)-hydrazono]-3-oxo-propionitrile (HJC0693); 3-(5-tert-Butyl-isoxazol-3-yl)-2-[(4-chlorophenyl)-hydrazono]-3-oxo-propionitrile (HJC0694); 3-(5-tert-Butyl-isoxazol-3-yl)-3-oxo-2-(phenyl-hydrazono)-propionitrile (HJC0695); 3-(5-tert-Butyl-isoxazol-3-yl)-2-[(2,5-dichlorophenyl)-hydrazono]-3-oxo-propionitrile (HJC0696); 3-(5-tert-Butyl-isoxazol-3-yl)-3-oxo-2-(m-tolyl-hydrazono)propionitrile (HJC0712); 3-(5-tert-Butyl-isoxazol-3-yl)-3-oxo-2-[(3-trifluoromethyl-phenyl)-hydrazono]propionitrile (HJC0720); 3-(5-tert-Butyl-isoxazol-3-yl)-2-[(3-nitrophenyl)-hydrazono]-3-oxo-propionitrile (HJC0721); 3-(5-tert-Butyl-isoxazol-3-yl)-3-oxo-2-(p-tolyl-hydrazono)propionitrile (HJC0724); 3-(5-tert-Butyl-isoxazol-3-yl)-2-[(3,5-dichlorophenyl)-hydrazono]-3-oxo-propionitrile (HJC0726); 2-[(4-Bromophenyl)-hydrazono]-3-(5-tert-butyl-isoxazol-3-yl)-3-oxo-propionitrile (HJC0742); 2-[(3-Bromophenyl)-hydrazono]-3-(5-tert-butyl-isoxazol-3-yl)-3-oxo-propionitrile (HJC0743); 3-(5-tert-Butyl-isoxazol-3-yl)-2-[(2,5-dimethylphenyl)-hydrazono]-3-oxo-propionitrile (HJC0744); 3-(5-tert-Butyl-isoxazol-3-yl)-3-oxo-2-(quinolin-6-yl-hydrazono)propionitrile (HJC0745); 3-(5-tert-Butyl-isoxazol-3-yl)-2-[(2,3-dichlorophenyl)-hydrazono]-3-oxo-propionitrile (HJC0750); 3-(5-tert-Butyl-isoxazol-3-yl)-2-[(3-ethynyl-phenyl)-hydrazono]-3-oxo-propionitrile (HJC0751); 3-{N-[2-(5-tert-Butyl-isoxazol-3-yl)-1-cyano-2-oxo-ethylidene]-hydrazino}benzoic acid ethyl ester (HJC0752); 3-{N-[2-(5-tert-Butyl-isoxazol-3-yl)-1-cyano-2-oxo-ethylidene]-hydrazino}benzonitrile (HJC0753); 2-[(3-Acetyl-phenyl)-hydrazono]-3-(5-tert-butyl-isoxazol-3-yl)-3-oxo-propionitrile (HJC0754); 3-(5-tert-Butyl-isoxazol-3-yl)-2-[(2,3-dimethylphenyl)-hydrazono]-3-oxo-propionitrile (HJC0755); 3-(5-tert-Butyl-isoxazol-3-yl)-2-[(3-hydroxymethylphenyl)-hydrazono]-3-oxo-propionitrile (HJC0756); 3-(5-tert-Butyl-isoxazol-3-yl)-2-(indan-5-yl-hydrazono)-3-oxo-propionitrile (HJC0757); 2-[(3,5-Bis-trifluoromethyl-phenyl)-hydrazono]-3-(5-tert-butyl-isoxazol-3-yl)-3-oxo-propionitrile (HJC0758); 2-{N-[2-(5-tert-Butyl-isoxazol-3-yl)-1-cyano-2-oxo-ethylidene]-hydrazino}-6-chloro-benzoic acid (HJC0759); 3-(5-tert-Butyl-isoxazol-3-yl)-2-[(3-chloro-4-hydroxy-phenyl)-hydrazono]-3-oxo-propionitrile (HJC0760); 2-[(3-Chloro-phenyl)-hydrazono]-3-(5-methyl-isoxazol-3-yl)-3-oxo-propionitrile (HJC0768); or 2-[(3,5-Dichlorophenyl)-hydrazono]-3-(5-methyl-isoxazol-3-yl)-3-oxo-propionitrile (HJC0770).

Certain embodiments are directed to using one or more EPAC modulators to treat or enhance a therapy for a disease or condition associated with EPAC activity.

Various chemical definitions related to EPAC modulating compounds are provided as follows.

As used herein, “predominantly one enantiomer” means that the compound contains at least 85% of one enantiomer, or more preferably at least 90% of one enantiomer, or even more preferably at least 95% of one enantiomer, or most preferably at least 99% of one enantiomer. Similarly, the phrase “substantially free from other optical isomers” means that the composition contains at most 5% of another enantiomer or diastereomer, more preferably 2% of another enantiomer or diastereomer, and most preferably 1% of another enantiomer or diastereomer. In certain aspects, one, both, or the predominant enantiomer forms or isomers are all covered.

As used herein, the term “nitro” means —NO₂; the term “halo” or “halogen” designates —F, —Cl, —Br or —I; the term “mercapto” means —SH; the term “cyano” means —CN; the term “azido” means —N₃; the term “silyl” means —SiH₃, and the term “hydroxy” means —OH.

The term “alkyl,” by itself or as part of another substituent, means, unless otherwise stated, a linear (i.e. unbranched) or branched carbon chain of 1, 2, 3, 4, 5, 6, 7, 8, 9, or 10 carbons, which may be fully saturated, monounsaturated, or polyunsaturated. An unsaturated alkyl group includes those having one or more carbon-carbon double bonds (alkenyl) and those having one or more carbon-carbon triple bonds (alkenyl). The groups, —CH₃(Me, methyl), —CH₂CH₃ (Et, ethyl), —CH₂CH₂CH₃ (n-Pr, n-propyl), —CH(CH₃)₂ (iso-Pr, iso-propyl), —CH₂CH₂CH₂CH₃ (n-Bu, n-butyl), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), are all non-limiting examples of alkyl groups.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O, S, and N. The heteroatom(s) may be placed at any interior position of the heteroalkyl group or at the position at which the alkyl group is attached to the remainder of the molecule. Up to two heteroatoms may be consecutive. The following groups are all non-limiting examples of heteroalkyl groups: trifluoromethyl, —CH₂F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The terms “cycloalkyl” and “heterocyclyl,” by themselves or in combination with other terms, means cyclic versions of “alkyl” and “heteroalkyl”, respectively. Additionally, for heterocyclyl, a heteroatom can occupy the position at which the heterocycle is attached to the remainder of the molecule. Examples of cycloalkyl groups include cyclopropyl, cyclobutyl, cyclopentyl, cyclohexyl, and cycloheptyl groups. Examples of heterocyclic groups include indole, azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane, tetrahydro-1,1-dioxothienyl, and the like.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 4-azaindole, 5-azaindole, 6-azaindole, 7-azaindole, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Examples of optional substituents include, but are not limited to: —OH, oxo (═O), —Cl, —F, —Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

The term “alkoxy” means a group having the structure —OR′, where R′ is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

The term “oxo” as used herein means oxygen that is double bonded to a carbon atom.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydro fluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base, such as methylamine, ethylamine, ethanolamine, lysine, ornithine and the like. Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable.

Additional examples of pharmaceutically acceptable salts and their methods of preparation and use are presented in Handbook of Pharmaceutical Salts: Properties, Selection and Use (2002), which is incorporated herein by reference.

An “isomer” of a first compound is a separate compound in which each molecule contains the same constituent atoms as the first compound, but where the three dimensional configuration of those atoms differs. Unless otherwise specified, the compounds described herein are meant to encompass their isomers as well. A “stereoisomer” is an isomer in which the same atoms are bonded to the same other atoms, but where the configuration of those atoms in three dimensions differs. “Enantiomers” are stereoisomers that are mirror images of each other, like left and right hands. “Diastereomers” are stereoisomers that are not enantiomers.

It is contemplated that any embodiment discussed in this specification can be implemented with respect to any method or composition of the invention, and vice versa. Furthermore, compositions of the invention can be used to achieve methods of the invention.

### II. PHARMACEUTICAL FORMULATIONS AND ADMINISTRATION

In certain embodiments, the invention also provides compositions comprising one or more EPAC modulator with one or more of the following: a pharmaceutically acceptable diluent; a carrier; a solubilizer; an emulsifier; a preservative; and/or an adjuvant. Such compositions may contain an effective amount of at least one EPAC modulator. Thus, the use of one or more EPAC modulators as provided herein for the preparation of a medicament is also included. Such compositions can be used in the treatment of a variety of EPAC associated diseases or conditions such as cancer or leptin associated disease or conditions.

An EPAC modulator may be formulated into therapeutic compositions in a variety of dosage forms such as, but not limited to, liquid solutions or suspensions, tablets, pills, powders, suppositories, polymeric microcapsules or microvesicles, liposomes, and injectable or infusible solutions. The preferred form depends upon the mode of administration and the particular disease targeted. The compositions also preferably include pharmaceutically acceptable vehicles, carriers, or adjuvants, well known in the art.

Acceptable formulation components for pharmaceutical preparations are nontoxic to recipients at the dosages and concentrations employed. In addition to the EPAC modulating agents, compositions may contain components for modifying, maintaining, or preserving, for example, the pH, osmolarity, viscosity, clarity, color, isotonicity, odor, sterility, stability, rate of dissolution or release, adsorption, or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 18 th Ed., (A. R. Gennaro, ed.), 1990, Mack Publishing Company), hereby incorporated by reference.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which may further include sorbitol or a suitable substitute therefor.

The pharmaceutical composition to be used for in vivo administration is typically sterile. Sterilization may be accomplished by filtration through sterile filtration membranes. If the composition is lyophilized, sterilization may be conducted either prior to or following lyophilization and reconstitution. The composition for parenteral administration may be stored in lyophilized form or in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

The above compositions can be administered using conventional modes of delivery including, but not limited to, intravenous, intraperitoneal, oral, intralymphatic, subcutaneous administration, intraarterial, intramuscular, intrapleural, intrathecal, and by perfusion through a regional catheter. Local administration to an organ or a tumor is also contemplated by the present invention. When administering the compositions by injection, the administration may be by continuous infusion or by single or multiple boluses. For parenteral administration, the EPAC modulating agents may be administered in a pyrogen-free, parenterally acceptable aqueous solution comprising the desired EPAC modulating agents in a pharmaceutically acceptable vehicle. A particularly suitable vehicle for parenteral injection is sterile distilled water in which one or more EPAC modulating agents are formulated as a sterile, isotonic solution, properly preserved.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

If desired, stabilizers that are conventionally employed in pharmaceutical compositions, such as sucrose, trehalose, or glycine, may be used. Typically, such stabilizers will be added in minor amounts ranging from, for example, about 0.1% to about 0.5% (w/v). Surfactant stabilizers, such as TWEEN®-20 or TWEEN®-80 (ICI Americas, Inc., Bridgewater, N.J., USA), may also be added in conventional amounts.

To determine the bioavailability of EPAC inhibitors, an IP injection formulation was developed in which the compounds were dissolved in ethanol and then diluted 1:10 with a 10% Tween 80 in normal saline solution. This formulation was determined suitable by passing the simulated in vivo blood dilution assay. In vivo pharmacokinetic studies were performed in four week old female C57BL6/N mice. Following one single intraperitoneal (IP) injection of the ESI-09 compound (10 mg/kg) in mice (n=5 for each time point), blood levels of ESI-09 were determined to be rapidly elevated reaching maximal values of 42,520 ng/ml (128 μM) at 0.5 hr with a half-life of 3.5 hrs. These results suggest that ESI-09 has an excellent bioactivity in vivo.

For the compounds of the present invention, alone or as part of a pharmaceutical composition, such doses are between about 0.001 mg/kg and 1 mg/kg body weight, preferably between about 1 and 100 μg/kg body weight, most preferably between 1 and 10 μg/kg body weight.

Therapeutically effective doses will be easily determined by one of skill in the art and will depend on the severity and course of the disease, the patient's health and response to treatment, the patient's age, weight, height, sex, previous medical history and the judgment of the treating physician.

In additional embodiments, patients may also be administered directly, endoscopically, intratracheally, intratumorally, intravenously, intralesionally, intramuscularly, intraperitoneally, regionally, percutaneously, topically, intrarterially, intravesically, or subcutaneously. Therapeutic compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

### III. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exchange Protein Directly Activated by cAMP Plays a Critical Role in Bacterial Invasion During Fatal Rickettsioses

The role of Epac1, a newly discovered family member of eukaryotic cAMP receptors, in the pathogenesis of rickettsiosis was studied using both genetic and pharmacological approaches in vivo. The rationale is twofold. First, cAMP signaling has been extensively manipulated by microbial pathogens to facilitate their virulence both from functions within the pathogens themselves and their mammalian host cells (McDonough and Rodriguez (2012) Nat Rev Microbiol 10(1):27-38). Second, Epac1 has been implicated as a key player in regulating various functions in endothelial cells, a major target of rickettsial infection. Based on Epac1's published functions in cell-cell junctions and barrier functions (Pannekoek et al. (2011) Cell Signal 23(12):2056-2064; Fukuhara et al. (2005) Mol Cell Biol 25(1):136-146; Kooistra et al. (2005) FEBS Lett 579(22):4966-4972; Schnoor et al. (2011) J Exp Med 208(8):1721-1735; Cullere et al. (2005) Blood 105(5):1950-1955; Spindler et al. (2011) Am J Pathol 179(4):1905-1916; Rampersad et al. (2010) J Biol Chem 285(44):33614-33622; Cheung et al. (2012) Am J Physiol Heart Circ Physiol 303(11):H1374-H1383), it was contemplated that deletion or inhibition of Epac1 in mice would compromise the endothelial barrier and lead to a more severe response to rickettsial infection. To the contrary, both genetic deletion and pharmacological inhibition of Epac1 protect mice from an ordinarily lethal dose of rickettsiae.

Rickettsiae-endothelial cell interactions are associated with several major steps, namely bacterial adhesion, invasion through induced phagocytosis, phagosomal escape for intracellular survival, bacterial replication, and motility enhancement for cell-to-cell spreading (Chan et al. (2010) Front Microbiol 1:139; Walker and Ismail (2008) Nat Rev Microbiol 6(5):375-386; Martinez et al. (2005) Cell 123(6):1013-1023; Martinez and Cossart (2004) J Cell Sci 117(Pt 21):5097-5106). Because Epac1 is known to regulate functions associated with cell adhesion and formation of intercellular junctions, particularly barrier functions in endothelial cells (Schmidt (2013) Pharmacol Rev 65(2):670-709), an ex vivo rickettsial infection model was developed to further identify at which step Epac1 plays a role in the bacteria-host cell interaction during rickettsioses. Studies using aortic rings prepared from WT C57BL/6 and Epac1−/−(null) mice allow the demonstration of deleting Epac1 prevents adhesion and/or invasion of rickettsiae into endothelial cells. This observation is further validated using ESI-09 and an in vitro rickettsial infection model that uses HUVECs (Gong et al. (2012) PLoS Negl Trop Dis 6(6):e1699). Considering the lack of effective animal models for many microbial pathogens, the establishment of aortic rings ex vivo as an endothelial infection model will impact not just rickettsiosis but also other studies of endothelial-related microbial pathogenesis. It represents a tool for examining cellular and molecular mechanisms of pathogen infection, as well as for testing potential treatments for endothelial-related microbial pathogenesis that would not be possible otherwise.

Rickettsiae induce their internalization into host cells by a receptor-mediated invasion mechanism using Ku70 as a potential host cell receptor for rickettsial autotransporter protein outer membrane protein B (OmpB) encoded by the surface cell antigen 5 (sca5) gene (Martinez et al. (2005) Cell 123(6):1013-1023; Chan et al. (2009) Cell Microbiol 11(4):629-644). Considering that Epac1 is capable of promoting the nuclear exit of DNA-PK in various cell types (Huston et al. (2008) Proc Natl Acad Sci USA 105(35):12791-1279647), it is conceivable that Epac1 may mediate rickettsial adhesion and/or invasion by modulating the cellular translocation of Ku70. However, in addition to Ku70, previous studies show that that entry of R. conorii into nonphagocytic mammalian cells also depends on actin and microtubule dynamics, the host endocytic machinery, and the activation of protein tyrosine kinases and lipid kinases (Martinez and Cossart (2004) J Cell Sci 117(21):5097-5106). Martinez and coworkers have reported that bacterial internalization correlates with cCbl-induced rapid ubiquitination of Ku70 and depletion of host clathrin and caveolin-2 inhibits OmpB-mediated rickettsial invasion of mammalian cells (Martinez (2005) Cell 123(6):1013-1023; Chan et al. (2009) Cell Microbiol 11(4):629-644). Recent data also suggest that another related rickettsial autotransporter protein, OmpA (encoded by sca0), is sufficient to mediate integrin-dependent invasion of mammalian cells (Hillman et al. (2013) Cell Microbiol 15(5):727-741). Epac1 is known to regulate all of the aforementioned cellular functions (Schmidt et al. (2013) Pharmacol Rev 65(2):670-709), but the precise molecular mechanism(s) by which Epac1 controls rickettsial adhesion and/or invasion is not clear at this time. Further research into understanding the signal crosstalk between Epac1 and endocytic pathways hijacked by rickettsiae is currently ongoing.

A. Results

Deletion of the Epac1 Gene in Mice Protects them from Fatal Rickettsiosis.

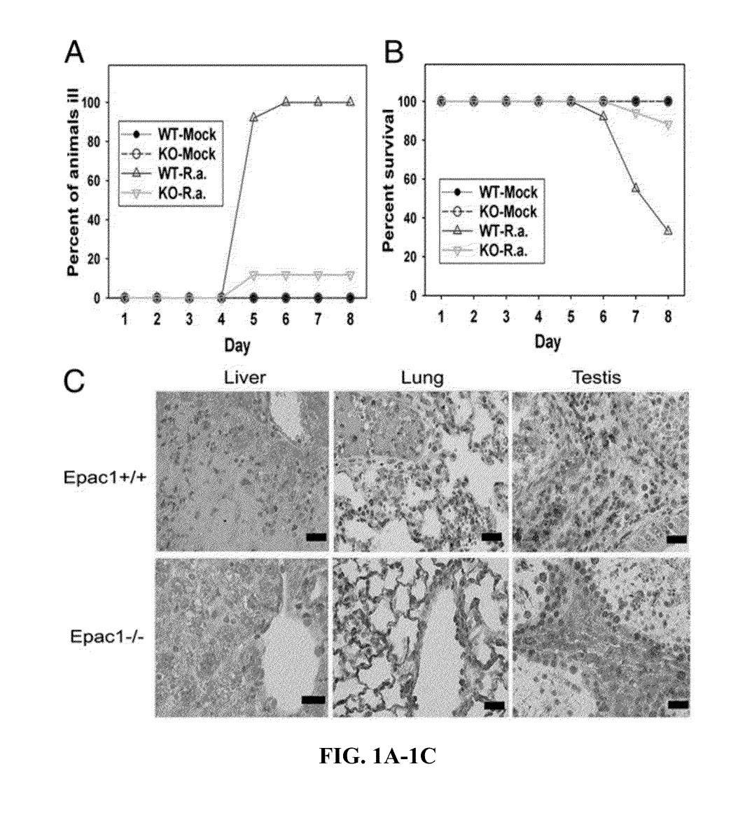

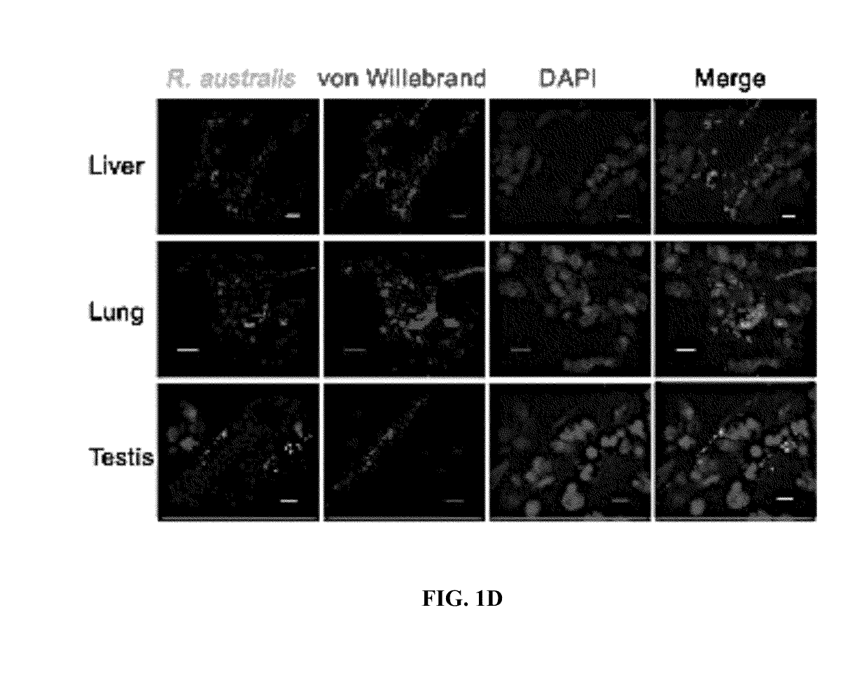

The functional role of Epac1 in rickettsioses was studied in an Epac1 knockout mouse model (Yan J, et al. (2013) Mol Cell Biol 33(5):918-926). The studies included challenging both Epac1−/− and C57BL/6 wild-type (WT) with Rickettsia australis. The C57BL/6 mouse-R. australis model is an established animal model of human spotted fever group (SFG) rickettsiosis because the pathology involves disseminated endothelial infection and pathological lesions, including vasculitis in multiple organs, similar to what is observed in human Rocky Mountain Spotted Fever (RMSF) (Feng et al. (1993) Am J Pathol 142(5):1471-1482; Xin et al. (2012) PLoS ONE 7(3):e34062). All of the WT mice became progressively ill starting on day 3 after inoculation, with typical signs of markedly ruffled fur, a hunched posture, and partially closed eyelids (FIG. 1A). Eight of 12 WT mice died by the end of the 8-d experiment (67% mortality) (FIG. 1B). Surprisingly, most of the Epac1−/− mice suffered only a very mild illness and only 2 of 17 Epac1−/− mice died (12% mortality). Consistent with the morbidity and mortality observations, histological examination of WT mice tissues revealed severe vasculitis and perivasculitis in the testes and liver, interstitial pneumonia, and multifocal hepatocellular necrosis (FIG. 1C). Immunofluorescent (IF) staining for rickettsial antigens and von Willebrand factor in tissue confirmed SFG rickettsiae infection in endothelial cells (FIG. 1D). Conversely, these typical pathological lesions associated with rickettsioses (Feng et al. (1993) Am J Pathol 142(5):1471-1482; Xin et al. (2012) PLoS ONE 7(3):e34062) were largely absent from the Epac1−/− mice given a dose of R. australis that normally kills at least half of the mice (FIG. 1C).

Rickettsial Infection Induces Increased Expression of Epac1 in Rickettsial Lesions.

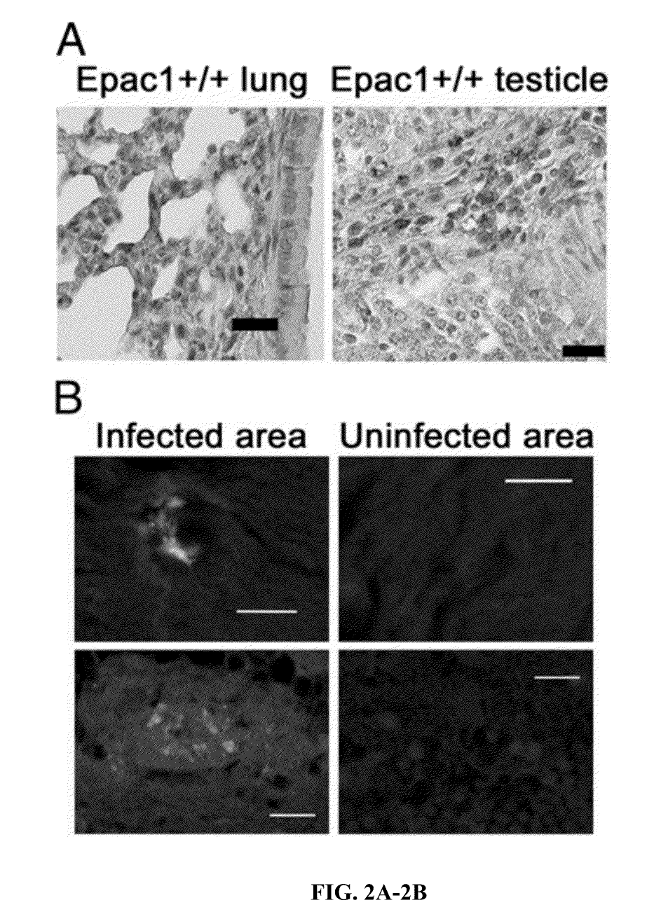

Expression profiling of endogenous Epac1 provided evidence that in WT C57BL/6 mice infection with R. australis induced increased expression of Epac1 in cells of the interstitium of mouse lung and testis where there are rickettsial antigen signals (FIG. 2A) 8 d after inoculation. Interestingly, dual-target IF studies on tissues from two cases of archival pediatric fatal RMSF also revealed increased Epac1 immunostaining signals within multiple rickettsial lesions located in the blood-brain barrier areas (FIG. 2B). These in vivo observations of enhanced expression of Epac1 within rickettsial lesions, coupled with the fact that deficiency of Epac1 protects mice against rickettsial infection, suggest that Epac1 plays a role in fatal SFG rickettsiosis.

Deletion of Epac1 Impedes Rickettsial Attachment and/or Invasion into the Endothelial Cells ex Vivo.

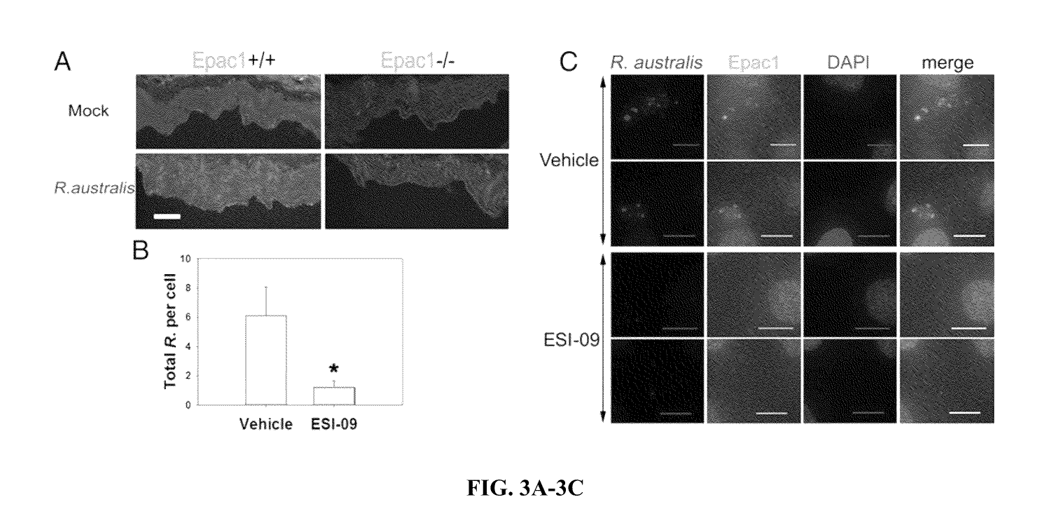

The molecular and cellular mechanisms by which Epac1 might be involved in rickettsial pathogenesis were determined. To determine whether lack of Epac1 directly impedes bacterial infection, an ex vivo vascular endothelial culture rickettsial infection model was established using aortic rings prepared from WT C57BL/6 and Epac1-null mice. Deletion of Epac1 nearly completely blocked rickettsial attachment and/or invasion into the endothelial layer of the aortic ring at 30 min post-exposure to R. australis at a dose of 1×10⁵ pfu per aortic ring while the endothelium of the aortic ring from WT mice was highly decorated by rickettsia (FIG. 3A).

Inhibition of Epac1 Blocks Rickettsial Attachment and Invasion into Nonphagocytic Host Cells.

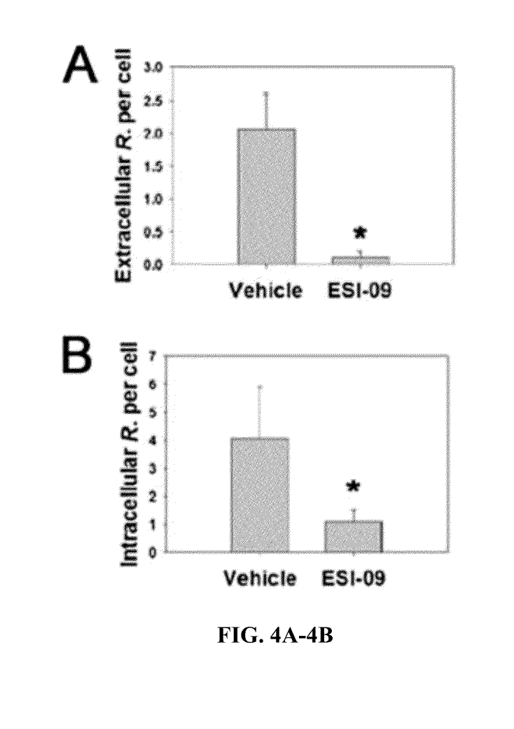

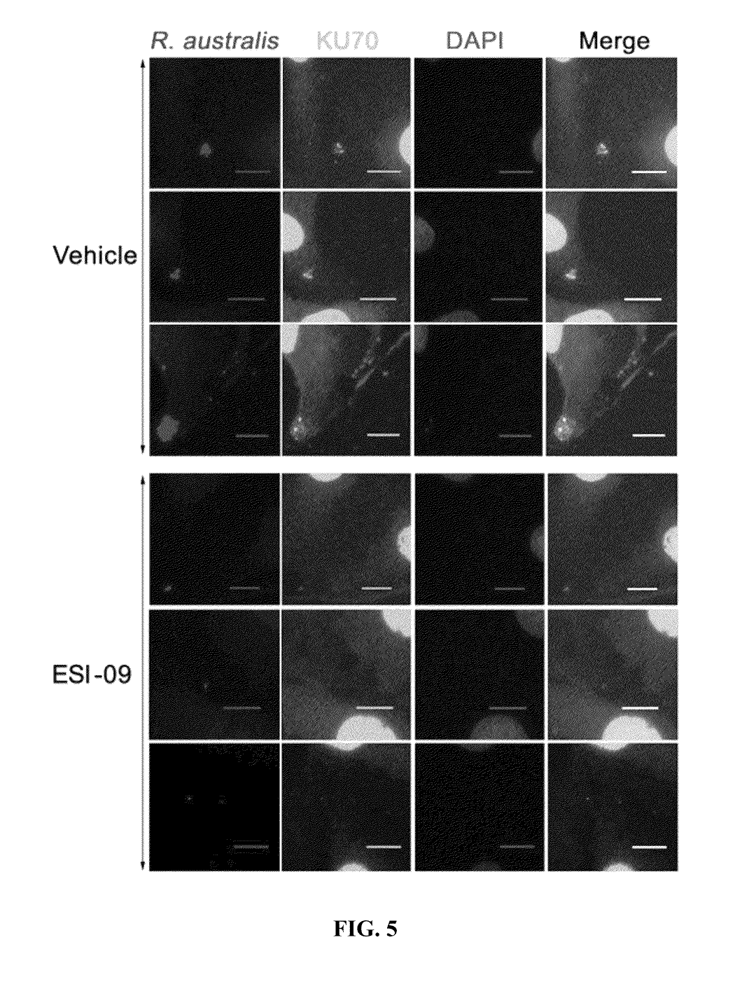

Taking advantage of a recently discovered Epac-specific inhibitor, ESI-09 (Tsalkova et al. (2012) Proc Natl Acad Sci USA 109(45):18613-18618; Almahariq et al. (2013) Mol Pharmacol 83(1):122-128), the rickettsial infection process in human umbilical vein endothelial cells (HUVECs) exposed to 5 μM ESI-09 or a vehicle sham were monitored. As shown in FIG. 3B and FIG. 4, exposure to ESI-09 significantly reduced intracellular and total bacterial counts in HUVECs at 30 min post-infection with 10 multiplicities of infection (MOI) of R. australis compared with similarly infected controls exposed to vehicle only. Moreover, IF staining revealed that Epac1 colocalized with rickettsiae (FIG. 3C) in the cytosol of HUVECs, whereas exposure to ESI-09 attenuated this interaction. An earlier investigation identified Ku70, a subunit of DNA-dependent protein kinase (DNA-PK), as a potential host cell receptor (Martinez et al. (2005) Cell 123(6):1013-1023). Indeed, IF analysis showed that Ku70 colocalized with the bacteria inside the cytosol of the HUVECs (FIG. 5). Again, pharmacological inhibition of Epac1 using ESI-09 significantly reduced such interactions. Taken together, these data suggest that Epac1 plays a critical role in the early stage of rickettsial attachment and invasion into non-phagocytic host cells.

Pharmacological Inhibition of Epac1 Protects WT Mice from Fatal SFG Rickettsiosis.

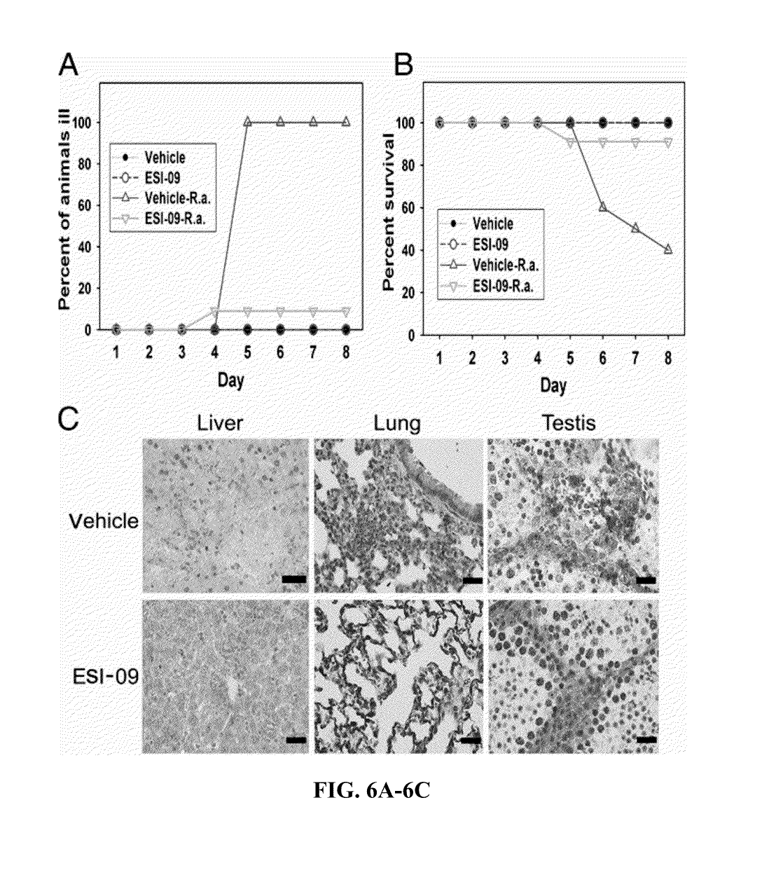

The use of Epac pharmacological intervention as a therapeutic strategy for combating fatal SFG rickettsioses was evaluated. WT C57BL/6 mice, randomly divided into two groups, were treated with ESI-09 (10 mg·kg⁻¹·d⁻¹) or vehicle via i.p. injection for 5 d, followed by i.v. inoculation of R. australis. ESI-09 treatment was continued for another 7 d. As shown in FIG. 6, treatment with ESI-09 dramatically protected WT mice against R. australis infection with much milder disease manifestations (FIG. 6A) and significantly improved survival (FIG. 6B). Only 1 of 11 ESI-09-treated mice died (9% mortality), compared with those of the vehicle-only group, in which 6 of 10 WT mice died (60% mortality) at the end of experiment. Histological evaluation confirmed that pharmacological inhibition of Epac significantly attenuated the pathological responses, resulting in milder vasculitis in the testis, occasional microvesicular hepatocellular fatty change, less interstitial inflammation in the lung, and immunohistochemical evidence of significantly less rickettsial antigen in tissues compared with the control group (FIG. 6C). Such observations from in vivo pharmacological inhibition of Epac recapitulated data based on genetic Epac1 knockout mice. Taken together, these results demonstrated that Epac1 inhibition is an effective therapeutic strategy for fatal SFG rickettsiosis.

B. Materials and Methods

Rickettsiae.

R. australis (Cutlack strain) was provided by C. Pretzman (Department of Health Laboratory, Columbus, Ohio) and was passaged three times in Vero cells (ATCC CCL81; American Type Culture Collection) and four times in embryonated chicken yolk sacs. For ex vivo and in vitro experiments, a 10% (vol/vol) yolk sac suspension of R. australis was propagated through two passages in Vero cells. R. australis were isolated using a bead-based protocol, as described previously (Gong et al. (2012) PLoS Negl Trop Dis 6(6):e1699). Purified rickettsiae were frozen in sucrose-phosphate-glutamate buffer at −80° C. (Gong et al. (2012) PLoS Negl Trop Dis 6(6):e1699; Xin et al. (2012) PLoS ONE 7(3):e34062). Rickettsial counts and infectivity of the frozen stocks were determined by plaque assay and tissue culture ID50 assays on Vero cells or HUVECs using established protocols (Gong et al. (2012) PLoS Negl Trop Dis 6(6):e1699; Xin et al. (2012) PLoS ONE 7(3):e34062; Hanson (1987) Am J Trop Med Hyg 36(3):631-638) and yielded ˜1×10⁹ infectious bacterial cells per milliliter. Uninfected Vero cells were processed by the same procedure as control material. The rickettsial stock was mycoplasma-free. All biosafety level (BSL) 3 or animal BSL3 experiments were performed in Centers for Disease Control and Prevention-certified facilities in the Galveston National Laboratory at the University of Texas Medical Branch (UTMB) using standard operating procedures and precautions.

Mice.

All animal experiments were performed according to protocols approved by the Institutional Animal Care and Use Committee of UTMB. C57BL/6 Epac1 null mice were derived as described previously (Yan et al. (2013) Mol Cell Biol 33(5):918-926). All mice used in this study were 8- to 12-wk-old males. C57BL/6 mice are highly susceptible to R. australis; therefore, this organism was chosen as the SFG rickettsial agent (Feng et al. (1993) Am J Pathol 142(5):1471-1482; Xin et al. (2012) PLoS ONE 7(3):e34062).

Rickettsial Challenge Study.

Experimental animals (12 WT and 17 Epac1−/− mice) were inoculated i.v. with 2×10⁶ pfu per mouse of R. australis and observed daily for illness and survival. Five WT and eight Epac1−/− mice were mock-infected and used as controls. Mice with markedly ruffled fur, a hunched posture, and partially closed eyelids were defined as severely ill and counted in the “ill” category (Feng et al. (1993) Am J Pathol 142(5):1471-1482; Xin et al. (2012) PLoS ONE 7(3):e34062).

Antibodies and Other Reagents.

Anti-Epac1 mouse monoclonal antibody (mAb) (5D3) was purchased from Cell Signaling. Anti-Ku70 mAb (Clone 162) was purchased from Thermo Scientific. A rabbit polyclonal antibody against SFG rickettsiae was described previously (Gong et al. (2012) PLoS Negl Trop Dis 6(6):e1699; Xin et al. (2012) PLoS ONE 7(3):e34062). The unconjugated Affini-Pure Fab fragment goat anti-mouse IgG (H+L) for immunohistochemistry (IHC) or IF using mouse tissues was purchased from Jackson ImmunoResearch Labs. Biotinylated goat anti-mouse and rabbit IgG, a fast red alkaline phosphatase substrate kit, and a 3,3′-diaminobenzidine (DAB) peroxidase substrate kit were purchased from Vector Laboratories. AlexaFluor 488- and AlexaFluor 594-conjugated goat anti-mouse and anti-rabbit IgG and ProLong Gold Antifade Reagent with DAPI were purchased from Invitrogen.

Histopathology and Immunohistology.

Complete necropsies were performed on all experimentally infected and control mice. Samples of liver, lung, and testis were fixed in a 4% (vol/vol) neutral buffered solution of formaldehyde, embedded in paraffin, sectioned at 5-μm thickness, and processed by staining with hematoxylin and eosin for evaluation of histopathology and by IHC or IF microscopy for detection of SFG rickettsiae and Epac1 as described previously (Gong et al. (2012) PLoS Negl Trop Dis 6(6):e1699). For IHC or IF studies on mouse tissue samples using mAbs, deparaffinized and rehydrated sections were blocked with unconjugated AffiniPure Fab fragment goat anti-mouse IgG (H+L) for 1 h at room temperature before incubation with mAb against Epac1 (dilution, 1:500) and rabbit polyclonal antibody against SFG rickettsiae (1:1,000) overnight at 4° C. Epac1 and rickettsiae were detected with biotinylated goat anti-mouse and anti-rabbit antibodies, respectively, followed by alkaline phosphatase-fast red and DAB reactions, respectively. For IF assays using archival human brain tissues, AlexaFluor 594 goat anti-mouse and AlexaFluor 488 goat anti-rabbit antibodies were used. Nuclei were counterstained with DAPI. Fluorescent images were analyzed using an Olympus BX51 epifluorescence or Olympus IX81 confocal microscope.

Ex Vivo Mouse Vascular Endothelial Assay for Rickettsial Infection.

An ex vivo vascular endothelial culture model for rickettsial infection was established using aortic rings isolated from three WT and three Epac1−/− mice. Briefly, aortae were first dissected from mice and then cleaned of adipose tissue and cut into five rings per mouse aorta. A total of 30 aortic rings were cultured for 48 h in Prigrow I medium (Applied Biological Materials) supplemented with 10% (vol/vol) FBS. Three rings per mouse were exposed to R. australis at 1×10⁵ pfu per ring in medium, and the other two rings were incubated in uninfected medium until microscopic evidence of neocellular growth was present after 1 wk to confirm the viability of the cultures. At the end of the experiment, all aortic rings were fixed in cold methanol for 24 h at −20° C., cryosectioned at 5-μm thickness, and processed using IF reagents to detect SFG rickettsiae and Epac1 as described above.

In Vitro Rickettsial Infection Assay. For in vitro studies, HUVECs (Cell Application) were cultivated in Prigrow I medium supplemented with 10% (vol/vol) heat-inactivated FBS in 5% (vol/vol) CO₂ at 37° C. All experiments were performed between passages 5 and 7, and cells were maintained in Prigrow I medium with 3% (vol/vol) FBS. Before infection with 10 MOI of rickettsiae, HUVECs were exposed to 5 μM EIS-09 in Prigrow I media for 1 h and were kept exposed at this concentration throughout the infection. For IF staining to detect rickettsiae, Epac1, and Ku70, the cells on the coverslips were washed extensively at least three times in PBS before the cells were fixed with cold methanol for 24 h and processed according to IF protocols detailed previously for ex vivo and in vivo models. Cells were examined and IF images were captured with an Olympus BX51 image system using a final 100× optical zoom. The number of HUVEC nuclei and total bacteria in each microscopic field were manually enumerated (Cardwell and Martinez (2012) Cell Microbiol 14(9):1485-1495; Riley et al. (2010) Infect Immun 78(5):1895-1904). The results were expressed as the ratio of R. australis organisms to HUVECs (nuclei) (Cardwell and Martinez (2012) Cell Microbiol 14(9):1485-1495; Riley et al. (2010) Infect Immun 78(5):1895-1904). Ten microscopic fields were examined for each experiment. Data are representative of at least three experiments. P values were determined using a standard Student t test.

Extracellular and total rickettsiae in host cell preparation were determined as described previously (Martinez and Cossart (2004) J Cell Sci 117(Pt 21):5097-5106; Cardwell and Martinez (2012) Cell Microbiol 14(9):1485-1495). Briefly, at the end of the experiment, HUVECs were washed extensively and fixed in 4% (vol/vol) paraformaldehyde at room temperature before being subjected to the above described IF staining procedure. For detection of extracellular bacterial signaling, fixed HUVECs were incubated with rabbit polyclonal antibody against SFG rickettsiae (1:1,000) for 2 h at room temperature and then incubated with AlexaFluor 594 goat anti-rabbit antibody. To detect total bacterial signaling, stained HUVECs were permeabilized in 0.1% Triton X-100 in PBS before reincubation with rabbit polyclonal antibody against SFG rickettsiae antibody and AlexaFluor 488 goat anti-rabbit antibody. Cells were examined and IF images were captured with an Olympus BX51 image system using a final 100× optical zoom. The number of HUVEC nuclei, extracellular adherent rickettsiae (detected by red fluorescence emission), and total rickettsiae (detected by green fluorescence emission) in each microscopic field were manually enumerated (Cardwell and Martinez (2012) Cell Microbiol 14(9):1485-1495; Riley et al. (2012) Infect Immun 80(8):2735-2743). Intracellular invaded rickettsiae were calculated subsequently (Martinez and Cossart (2004) J Cell Sci 117(Pt 21):5097-5106; Cardwell and Martinez (2012) Cell Microbiol 14(9):1485-1495). Ten microscopic fields were examined for each experiment. The results were expressed as the ratio of R. australis organisms to HUVECs (nuclei) per examined field (Cardwell and Martinez (2012) Cell Microbiol 14(9):1485-1495; Riley et al. (2012) Infect Immun 80(8):2735-2743; Read et al. (2012) Cell Microbiol 14(4):529-545). Data are representative of at least three experiments. P values were determined using a standard Student t test.

In Vivo ESI-09 Treatment of R. australis-Infected Epac1+/+Mice.

Thirty-three WT C57BL/6 mice were divided into four groups [11 mice (group A), 10 mice (group B), 6 mice each (groups C and D)]. Groups A and C were treated with the Epac-specific inhibitor ESI-09 [10 mg·kg⁻¹·d⁻¹ dissolved in buffered saline containing 10% (vol/vol) ethanol and 10% (vol/vol) Tween-80] via i.p. injection for 5 d before infection, whereas groups B and D were treated with vehicle, followed by i.v. inoculation of R. australis (2×10⁶ pfu per mouse) for groups A and B or mock inoculation for groups C and D. ESI-09 or vehicle treatment was continued for another 7 d until mice were killed on day 8. During the course of the experiments, animals were monitored daily for signs of illness and mortality.

1. A method for treating Rickettsia infection comprising administering an EPAC inhibitor to a subject having or at risk of having a Rickettsia infection.

2. The method of claim 1, wherein the EPAC inhibitor is N-(5-tert-Butyl-isoxazol-3-yl)-2-[(3-chlorophenyl)-hydrazono]-2-cyanoacetamide (HJC0683); 2-[(3-Chlorophenyl)-hydrazono]-2-cyano-N-(5-methyl-isoxazol-3-yl)acetamide (HJC0692); 3-(5-tert-Butyl-isoxazol-3-yl)-2-[(3-chlorophenyl)-hydrazono]-3-oxo-propionitrile (ESI-09); 3-(5-tert-Butyl-isoxazol-3-yl)-2-[(2-chlorophenyl)-hydrazono]-3-oxo-propionitrile (HJC0693); 3-(5-tert-Butyl-isoxazol-3-yl)-2-[(4-chlorophenyl)-hydrazono]-3-oxo-propionitrile (HJC0694); 345-tert-Butyl-isoxazol-3-yl)-3-oxo-2-(phenyl-hydrazono)-propionitrile (HJC0695); 3-(5-tert-Butyl-isoxazol-3-yl)-2-[(2,5-dichlorophenyl)-hydrazono]-3-oxo-propionitrile (HJC0696); 3-(5-tert-Butyl-isoxazol-3-yl)-3-oxo-2-(m-tolyl-hydrazono)propionitrile (HJC0712); 3-(5-tert-Butyl-isoxazol-3-yl)-3-oxo-2-[(3-trifluoromethyl-phenyl)-hydrazono]propionitrile (HJC0720); 3-(5-tert-Butyl-isoxazol-3-yl)-2-[(3-nitrophenyl)-hydrazono]-3-oxo-propionitrile (HJC0721); 3-(5-tert-Butyl-isoxazol-3-yl)-3-oxo-2-(p-tolyl-hydrazono)propionitrile (HJC0724); 3-(5-tert-Butyl-isoxazol-3-yl)-2-[(3,5-dichlorophenyl)-hydrazono]-3-oxo-propionitrile (HJC0726); 2-[(4-Bromophenyl)-hydrazono]-3-(5-tert-butyl-isoxazol-3-yl)-3-oxo-propionitrile (HJC0742); 2-[(3-Bromophenyl)-hydrazono]-3-(5-tert-butyl-isoxazol-3-yl)-3-oxo-propionitrile (HJC0743); 3-(5-tert-Butyl-isoxazol-3-yl)-2-[(2,5-dimethylphenyl)-hydrazono]-3-oxo-propionitrile (HJC0744); 3-(5-tert-Butyl-isoxazol-3-yl)-3-oxo-2-(quinolin-6-yl-hydrazono)propionitrile (HJC0745); 3-(5-tert-Butyl-isoxazol-3-yl)-2-[(2,3-dichlorophenyl)-hydrazono]-3-oxo-propionitrile (HJC0750); 3-(5-tert-Butyl-isoxazol-3-yl)-2-[(3-ethynyl-phenyl)-hydrazono]-3-oxo-propionitrile (HJC0751); 3-{N-[2-(5-tert-Butyl-isoxazol-3-yl)-1-cyano-2-oxo-ethylidene]-hydrazino}benzoic acid ethyl ester (HJC0752); 3-{N-[2-(5-tert-Butyl-isoxazol-3-yl)-1-cyano-2-oxo-ethylidene]-hydrazino}benzonitrile (HJC0753); 2-[(3-Acetyl-phenyl)-hydrazono]-3-(5-tert-butyl-isoxazol-3-yl)-3-oxo-propionitrile (HJC0754); 3-(5-tert-Butyl-isoxazol-3-yl)-2-[(2,3-dimethylphenyl)-hydrazono]-3-oxo-propionitrile (HJC0755); 3-(5-tert-Butyl-isoxazol-3-yl)-2-[(3-hydroxymethylphenyl)-hydrazono]-3-oxo-propionitrile (HJC0756); 3-(5-tert-Butyl-isoxazol-3-yl)-2-(indan-5-yl-hydrazono)-3-oxo-propionitrile (HJC0757); 2-[(3,5-Bis-trifluoromethyl-phenyl)-hydrazono]-3-(5-tert-butyl-isoxazol-3-yl)-3-oxo-propionitrile (HJC0758); 2-{N-[2-(5-tert-Butyl-isoxazol-3-yl)-1-cyano-2-oxo-ethylidene]-hydrazino}-6-chloro-benzoic acid (HJC0759); 3-(5-tert-Butyl-isoxazol-3-yl)-2-[(3-chloro-4-hydroxy-phenyl)-hydrazono]-3-oxo-propionitrile (HJC0760); 2-[(3-Chloro-phenyl)-hydrazono]-3-(5-methyl-isoxazol-3-yl)-3-oxo-propionitrile (HJC0768); or 2-[(3,5-Dichlorophenyl)-hydrazono]-3-(5-methyl-isoxazol-3-yl)-3-oxo-propionitrile (HJC0770).

3. The method of claim 1, wherein the EPAC inhibitor is 3-(5-tert-Butyl-isoxazol-3-yl)-2-[(3-chlorophenyl)-hydrazono]-3-oxo-propionitrile (ESI-09) or 3-(5-tert-Butyl-isoxazol-3-yl)-2-[(3,5-dichlorophenyl)-hydrazono]-3-oxo-propionitrile (HJC0726).

4. The method of claim 1, wherein the rickettsia infection is a Rickettsia prowazekii, Rickettsia typhi, or Rickettsia rickettsii infection.

5. The method of claim 1, further comprising administering a second therapeutic agent.

6. The method of claim 5, wherein the second therapeutic agent is an antigen or a therapeutic antibody.
